(19) United States
(12) Patent Application Publication
(10) Pub. No.: US 2025/0143791 A1
(43) Pub. Date: May 8, 2025

(54) SYSTEMS, DEVICES AND METHODS FOR TARGETED TISSUE THERAPY

(71) Applicant: LIGHT MATTER INTERACTION INC., ETOBICOKE (CA)

(72) Inventors: DARREN KRAEMER, TORONTO (CA); R.J. DWAYNE MILLER, PORT CREDIT (CA)

(21) Appl. No.: 18/838,305

(22) PCT Filed: Jul. 11, 2023
(86) PCT No.: PCT/CA2023/050929

§ 371 Date: Aug. 14, 2024

Related U.S. Application Data
(60) Provisional application No. 63/388,136, filed on Jul. 11, 2022.

(51) Int. Cl. A61B 18/22; A61B 18/00; A61B 18/20; A61B 34/00; A61B 34/20

(52) U.S. Cl. CPC A61B 18/22; A61B 34/20; A61B 34/25; A61B 2018/00202; A61B 2018/00702; A61B 2018/00761; A61B 2018/2005; A61B 2018/2035; A61B 2018/2211; A61B 2034/2063; A61B 2218/007

(57) ABSTRACT

Systems and methods are disclosed that facilitate the local therapy of tissue within the body. In some example embodiments, infrared laser pulses are locally delivered, via optical fiber, to an intracorporal target tissue region and are provided with pulse conditions suitable for causing local tissue disruption and liquification, leading to fine tissue disruption, tissue homogenization, and removal of vasculature and interstitial fluid channels, and enabling passage of the distal tip of the optical fiber into the target tissue region without substantial tissue deformation and damage along a preferred surgical pathway. When optical fiber emitting such pulses is employed to penetrate tumor tissue, the resulting reduction of interstitial fluid pressure facilitates the subsequent injection of a drug into the tumor, enabling the drug to remain localized within the tumor with reduced diffusion. The tumor disruption and subsequent drug delivery may be performed using an integrated optical and fluidic delivery device.

This application claims priority to U.S. Provisional Patent Application No. 63/388,136, titled “SYSTEMS, DEVICES AND METHODS FOR TARGETED DRUG DELIVERY” and filed on Jul. 11, 2022, the entire contents of which is incorporated herein by reference.

### BACKGROUND

The present disclosure relates to systems and methods for delivering local therapy to a target tissue region inside of the body.

Cancer treatments involve a plurality of treatment regimes. One of the most often used treatments is the use of anticancer drugs in a procedure known as chemo treatments. These drugs are administered systemically due to the challenges of directly injecting drugs at cancer sites. This requirement means that cancer drugs must be highly selective for uptake by rapidly dividing cells, or other morphological features, that are unique to cancer cells (or other diseased tissue) over normal cells.

Unfortunately, in solid tumors, several factors inhibit the homogenous distribution of systemic drugs, including limited regional blood flow to the tumor, permeability of the tumor vasculature, structural barriers imposed by perivascular tumor cells and extracellular matrix, and intratumoral pressure. These impediments to the delivery of systemic drugs to tumor can be understood from FIG. 1 of Kobayashi et al. (Kobayashi H, Watanabe R, Choyke P L. Improving Conventional Enhanced Permeability and Retention (EPR) Effects; What Is the Appropriate Target?, Theranostics 2014; 4(1):81-89. doi:10.7150/thno.7193), which shows the local tumor environment and morphology. The impact of the local tumor morphology and pressure can greatly limit the number of drugs that can be successfully delivered. Moreover, even in the case of highly selective drugs, there are still major problems with the side effects of chemotherapy. The quality of life for people enduring chemotherapy can be extremely poor and can require life support in many cases.

As shown in FIG. 1A of Böckelmann et al. (Clemens Böckelmann & Udo Schumacher (2019): Targeting tumor interstitial fluid pressure: will it yield novel successful therapies for solid tumors?, Expert Opinion on Therapeutic Targets, DOI: 10.1080/14728222.2019.1702974), in normal tissue, capillaries are covered by pericytes, the surrounding interstitium contains loose extracellular matrix (ECM) produced by local fibroblasts and penetrated by lymphatic vessels. Hydrostatic and colloid osmotic pressures illustrate the opposing forces involved in filtration and transvascular fluid exchange; in steady state net outward filtration pressure is around 1 mm Hg. The distribution of solute molecules by diffusion covers for a distance of up to 100 μm from the capillary (dashed line). Convection flow reaches out of capillaries, through the interstitial space, and into the lymphatic vessels and is based upon a positive pressure gradient between the capillary and the lymphatic vessels.

However, as shown in FIG. 1B of Böckelmann et al., in tumor tissue, high interstitial fluid pressure (IFP) reverses the situation resulting in a net increase in the pressure within the interior of the tumor, ultimately hampering convection driven transport of solute molecules. The pressure gradient is now reaching from the center toward the periphery thereby enabling an opposite fluid flow. This leads to a gradient of decreasing oxygen (O₂), nutrient, and drug uptake. IFP is a product of a leaky tumor vasculature, missing lymph vessels, and a denser and stiffer ECM produced by cancer-associated fibroblasts (CAFs).

### SUMMARY

Systems and methods are disclosed that facilitate the local therapy of tissue within the body. In some example embodiments, infrared laser pulses are locally delivered, via optical fiber, to an intracorporal target tissue region and are provided with pulse conditions suitable for causing local tissue disruption and liquification, leading to fine tissue disruption, tissue homogenization, and removal of vasculature and interstitial fluid channels, and enabling passage of the distal tip of the optical fiber into the target tissue region without substantial tissue deformation and damage along a preferred surgical pathway. When an optical fiber emitting such pulses is employed to penetrate tumor tissue, the resulting reduction of interstitial fluid pressure facilitates the subsequent injection of a drug into the tumor, enabling the drug to remain localized within the tumor with reduced diffusion. The tumor disruption and subsequent drug delivery may be performed using an integrated optical and fluidic delivery device.

Accordingly, in one aspect, there is provided a system for performing local tissue disruption and liquification of an intracorporeal tissue region, the system comprising:
a pulsed infrared laser source configured to generate infrared laser pulses;
a laser pulse delivery assembly comprising:
   an optical fiber optically coupled to the pulsed infrared laser source, such that the infrared laser pulses are delivered through the optical fiber to a distal tip of the optical fiber; and
   a cannula configured to receive and mechanically support the optical fiber, such that the distal tip of the optical fiber is extendable to at least a distal end of the cannula for delivering the infrared laser pulses beyond the distal end of the cannula;
a navigation system configured to provide guidance during manipulation of the laser pulse delivery assembly to position the distal tip of the optical fiber proximal to the intracorporeal tissue region; and
control circuitry operatively coupled to the pulsed infrared laser source, the control circuitry being configured to perform operations comprising:
   controlling the pulsed infrared laser source to emit the infrared laser pulses such that during penetration of the intracorporeal tissue region by the distal tip of the optical fiber, the infrared laser pulses have laser pulse properties including:
      a wavelength selected such that absorption by a laser-irradiated tissue volume is predominantly due to excitation of vibrational modes of one or more constituents of the laser-irradiated tissue volume;
      a pulse duration that is shorter than a first time duration required for thermal diffusion out of the laser-irradiated tissue volume and shorter than a second time duration required for a thermally driven expansion of the laser-irradiated tissue volume;
      a pulse fluence and the pulse duration resulting in a peak pulse intensity below a threshold for ionization-driven tissue disruption and liquification to occur within the laser-irradiated tissue volume; and
      wherein the pulse fluence is sufficiently high to cause local tissue disruption and liquification of the laser-irradiated tissue volume;
   the pulsed infrared laser source thereby being controlled to facilitate local tissue disruption and liquification during penetration of the distal tip of the optical fiber into the intracorporeal tissue region, avoiding substantial deformation of the intracorporeal tissue region and facilitating positioning of the distal tip within the intracorporeal tissue region.

In some example implementations of the system, the control circuitry is configured to control the pulsed infrared laser source to deliver the infrared laser pulses with the laser pulse properties during manipulation of the laser pulse delivery assembly to position the distal tip of the optical fiber proximal to the intracorporeal tissue region, thereby locally disrupting tissue residing adjacent to the distal tip of the optical fiber while the distal tip of the optical fiber is moved through tissue toward the intracorporeal tissue region, avoiding substantial tissue deformation and facilitating positioning of the distal tip proximal to the intracorporeal tissue region.

In some example implementations of the system, the distal tip of the optical fiber is extendable beyond the distal end of the cannula to facilitate penetration of the intracorporeal tissue region by the distal tip of the optical fiber.

In some example implementations of the system, the control circuitry is further configured, after extension of the distal tip of the optical fiber into the intracorporeal tissue region, to control the pulsed infrared laser source to emit the infrared laser pulses with a reduced pulse fluence below a threshold for local tissue disruption and liquification, the reduced pulse fluence being sufficiently high to deliver thermal therapy within the intracorporeal tissue region for inducing apoptosis.

In some example implementations, the system further comprises an additional laser source optically coupled to the optical fiber, the additional laser source being configured to generate laser energy suitable for providing thermal therapy to the intracorporeal tissue region, wherein the control circuitry is further configured, after extension of the distal tip of the optical fiber into the intracorporeal tissue region, to control the additional laser source to emit the laser energy for inducing apoptosis within the intracorporeal tissue region.

In some example implementations, the system further comprises an optical detection system optically coupled to the optical fiber, the optical detection system being configured to deliver interrogating optical energy to tissue disrupted and liquified by the infrared laser pulses, and to detect optical energy responsively emitted by the disrupted and liquified tissue.

In some example implementations of the system, the laser pulse delivery assembly further comprises a liquid delivery conduit in flow communication with the distal end of the cannula; the system further comprising a liquid delivery pump configured to deliver a liquid therapeutic agent to the liquid delivery conduit; and wherein the control circuitry is operatively coupled to the liquid delivery pump, and wherein the control circuitry is further configured, after extension of the distal end of the cannula into the intracorporeal tissue region, to perform operations comprising: controlling the liquid delivery pump to dispense the liquid therapeutic agent within the intracorporeal tissue region.

The cannula may comprise a primary lumen through which the optical fiber is extendable, and wherein the liquid delivery conduit is provided as a side lumen of the cannula, the side lumen intersecting the primary lumen at an internal port residing within a distal region of the cannula, such that the liquid therapeutic agent residing in the liquid delivery conduit is brought into flow communication with the primary lumen, for dispensing the liquid therapeutic agent beyond the distal end of the cannula, after retraction of the distal tip of the optical fiber to a location that is proximal relative to the internal port.

The control circuitry may be configured to control the liquid delivery pump to deliver the liquid therapeutic agent within the intracorporeal tissue region after having previously delivered thermal therapy to the intracorporeal tissue region.

In some example implementations of the system, the liquid therapeutic agent comprises a photodynamic therapy agent, the system further comprising a photodynamic excitation laser source optically coupled to the optical fiber, the photodynamic excitation laser source being configured to generate photodynamic laser energy suitable for causing photodynamic activation of the photodynamic therapy agent, wherein the control circuitry is further configured, after dispensing of the liquid therapeutic agent into the intracorporeal tissue region, to control the photodynamic excitation laser source to emit the photodynamic laser energy for activating the photodynamic therapy agent.

In some example implementations of the system, the laser pulse delivery assembly further comprises a microbiopsy aspiration conduit in flow communication with a lumen of the cannula; the system further comprising a microbiopsy aspiration pump configured to cause a reduction in pressure within the microbiopsy aspiration conduit; and wherein the control circuitry is operatively coupled to the microbiopsy aspiration pump, and wherein the control circuitry is further configured, after extension of the distal end of the cannula into the intracorporeal tissue region and local disruption and liquification of tissue within the intracorporeal tissue region, to perform operations comprising: controlling the microbiopsy aspiration pump to aspirate a liquified tissue sample within the lumen of the cannula.

In some example implementations of the system, the laser pulse delivery assembly further comprises an aspiration conduit in flow communication with a distal region of the cannula; the system further comprising an aspiration pump configured to cause a reduction in pressure within the aspiration conduit; and wherein the control circuitry is operatively coupled to the aspiration pump, and wherein the control circuitry is further configured, during local disruption and liquification of tissue, to perform operations comprising: controlling the aspiration pump to aspirate liquified tissue within the aspiration conduit.

In some example implementations of the system, the navigation system comprises an ultrasound imaging system, and wherein the ultrasound imaging system is configured to display, on a user interface, a location of the distal tip of the optical fiber, the location being determined based on detection of photoacoustic signals generated at the distal tip during delivery of the infrared laser pulses with the laser pulse properties.

In some example implementations of the system, a distal region of the cannula is tapered, such that an outer diameter of the cannula reduces in a distal direction toward the distal end of the cannula.

In some example implementations of the system, a diameter of the cannula, at the distal end of the cannula, exceeds a diameter of the optical fiber by less than 10% of the diameter of the optical fiber.

In some example implementations of the system, the distal end of the cannula is beveled.

In some example implementations of the system, the distal tip of the optical fiber is beveled, such that the infrared laser pulses are emitted at an oblique angle relative to a longitudinal axis of the optical fiber. A bevel angle of the optical fiber may lie within 10% of a bevel angle of the distal end of the cannula.

In some example implementations of the system, the optical fiber is rotatable relative to the intracorporeal tissue region, and wherein the control circuitry is further configured, after extension of the distal tip of the optical fiber into the intracorporeal tissue region, to control the pulsed infrared laser source to emit the infrared laser pulses with the laser pulse properties during rotation of the optical fiber to facilitate local disruption and liquification over an expanded volume within the intracorporeal tissue region.

In some example implementation, the system further comprises a steering means for steering one or both of the cannula and the optical fiber.

In some example implementations of the system, the laser pulse delivery assembly comprises one or more additional optical fibers, such that the optical fiber and the one or more additional optical fibers form an optical fiber bundle, and wherein the optical fiber bundle is optically coupled to the pulsed infrared laser source, such that the infrared laser pulses are delivered through the optical fiber bundle to a distal end of the optical fiber bundle.

In some example implementations of the system, at least two optical fibers of the optical fiber bundle have beveled distal tips configured to direct the infrared laser pulses in different directions.

In another aspect, there is provided a method of performing local tissue disruption and liquification of an intracorporeal tissue region, the method comprising:
while extending an optical fiber within a body, such that a distal tip of the optical fiber penetrates an intracorporeal tissue region, delivering, through the optical fiber, infrared laser pulses having laser pulse properties including:
   a wavelength selected such that absorption by a laser-irradiated tissue volume is predominantly due to excitation of vibrational modes of one or more constituents of the laser-irradiated tissue volume;
   a pulse duration that is shorter than a first time duration required for thermal diffusion out of the laser-irradiated tissue volume and shorter than a second time duration required for a thermally driven expansion of the laser-irradiated tissue volume;
   a pulse fluence and the pulse duration resulting in a peak pulse intensity below a threshold for ionization-driven tissue disruption and liquification to occur within the laser-irradiated tissue volume; and wherein the pulse fluence is sufficiently high to cause local tissue disruption and liquification of the laser-irradiated tissue volume;
the infrared laser pulses thereby causing local tissue disruption and liquification during penetration of the distal tip of the optical fiber into the intracorporeal tissue region, avoiding substantial deformation of the intracorporeal tissue region and facilitating positioning of the distal tip within the intracorporeal tissue region.

In some example implementations of the method, prior to penetrating the intracorporeal tissue region, the infrared laser pulses are delivered during manipulation of the optical fiber to position the distal tip of the optical fiber proximal to the intracorporeal tissue region, thereby locally disrupting tissue residing adjacent to the distal tip of the optical fiber while the distal tip of the optical fiber is moved through tissue toward the intracorporeal tissue region, avoiding substantial tissue deformation and facilitating positioning of the distal tip proximal to the intracorporeal tissue region.

In some example implementations of the method, after extension of the distal tip of the optical fiber into the intracorporeal tissue region, additional infrared laser pulses with a reduced pulse fluence below a threshold for local tissue disruption and liquification are delivered by the optical fiber, the reduced pulse fluence being sufficiently high to deliver thermal therapy within the intracorporeal tissue region for inducing apoptosis.

In some example implementation the method further comprises employing an additional laser source optically coupled to the optical fiber, to deliver laser energy suitable for providing thermal therapy to the intracorporeal tissue region to induce apoptosis within the intracorporeal tissue region.

In some example implementation the method further comprises employing an optical detection system optically coupled to the optical fiber to deliver interrogating optical energy to tissue disrupted and liquified by the infrared laser pulses, and to detect optical energy responsively emitted by the disrupted and liquified tissue.

In some example implementations of the method, the distal tip of the optical fiber is extended beyond a distal end of a cannula to facilitate penetration of the intracorporeal tissue region.

The cannula may further comprise a liquid delivery conduit in flow communication a distal end of the cannula, the liquid delivery conduit being primed with a liquid therapeutic agent, the method further comprising, after having penetrated the intracorporeal tissue region with the distal tip of the optical fiber: extending the distal end of the cannula into the intracorporeal tissue region; retracting the optical fiber into the cannula; and dispensing the liquid therapeutic agent within the intracorporeal tissue region.

The cannula may comprise a primary lumen through which the optical fiber is extendable, and wherein the liquid delivery conduit is provided as a side lumen of the cannula, the side lumen intersecting the primary lumen at an internal port residing within a distal region of the cannula, such that the liquid therapeutic agent residing in the liquid delivery conduit is brought into flow communication with the primary lumen, for dispensing the liquid therapeutic agent beyond the distal end of the cannula, after retraction of the distal tip of the optical fiber to a location that is proximal relative to the internal port.

The method may further comprise delivering the liquid therapeutic agent within the intracorporeal tissue region after having previously delivered thermal therapy to the intracorporeal tissue region.

In some example implementations of the method, the liquid therapeutic agent comprises a photodynamic therapy agent, the method further comprising employing a photodynamic excitation laser source optically coupled to the optical fiber to deliver photodynamic laser energy suitable for causing photodynamic activation of the photodynamic therapy agent.

In some example implementations, the method further comprises, after having penetrated the intracorporeal tissue region with the distal tip of the optical fiber: extending the distal end of the cannula into the intracorporeal tissue region; employing a pump to lower a pressure in a lumen of the cannula to aspirate a liquified tissue sample.

In some example implementations of the method, penetration of the intracorporeal tissue region by the optical fiber is guided by an ultrasound imaging system, and wherein the ultrasound imaging system is configured to display, on a user interface, a location of the distal tip of the optical fiber, the location being determined based on detection of photoacoustic signals generated at the distal tip during delivery of the infrared laser pulses with the laser pulse properties.

In some example implementations of the method, the distal tip of the optical fiber is beveled, such that the infrared laser pulses are emitted at an oblique angle relative to a longitudinal axis of the optical fiber.

In some example implementations, the method further comprises, after extension of the distal tip of the optical fiber into the intracorporeal tissue region, emitting additional infrared laser pulses with the laser pulse properties during rotation of the optical fiber to facilitate local disruption and liquification over an expanded volume within the intracorporeal tissue region.

In some example implementations of the method, the intracorporeal tissue region is a tumor.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates the high Interstitial fluid pressure inside a solid tumor, requiring high injection force, and leading to a net outflow of drugs.

FIG. 2 plots the threshold fluence, as a function of wavelength, for ionization and for tissue disruption and liquification via the Pulsed InfraRed Laser (PIRL) impulsive heat deposition tissue disruption mechanism. Pulse durations may be controlled, depending on tissue type, to satisfy complete energy confinement to drive tissue disruption for extraction and reduction of interstitial fluid pressure in the targeted tissue. PIRL represents one example approach to achieve these conditions.

FIG. 3A illustrates the deformation of tumor tissue when attempting to penetrate the tumoral tissue with a conventional needle (beveled cannula).

FIG. 3B illustrates the penetration of tumor tissue, in the absence of substantial deformation of the tumor tissue, by an optical fiber that delivers PIRL pulses that produce local disruption and liquification of the tissue.

FIG. 4 illustrates an example system for PIRL-based local tissue disruption and thermal therapy.

FIG. 5 illustrates the use of an optical fiber to achieve intratumoral access via the delivery of PIRL laser pulses during translation of the optical fiber into the tumor and the subsequent delivery of thermal therapy within the tumor.

FIGS. 6A and 6B schematically illustrate the direct injection of a liquid therapeutic agent into a tumor via initial pulsed infrared laser pulse mediated tissue disruption followed by direct drug injection into the disrupted tissue.

FIG. 7 illustrates an example system for PIRL-based local tissue disruption and drug delivery.

FIGS. 8A, 8B, 8C and 8D illustrate an example cannula having a central lumen for advancement and retraction of an optical fiber, and an integrated fluidic channel for controllable drug delivery.

FIG. 9 illustrates an example distal fiber optical tip having a beveled surface.

FIG. 10 illustrates beam steering of one or more optical fibers having beveled distal surfaces.

FIG. 11 illustrates an example distal fiber optical tip having a movable component for directional steering.

FIG. 12 illustrates an example embodiment in which a hollow optical fiber is provided with a removable end piece for controlled fluid delivery.

FIG. 13 shows an example PIRL laser or other system with sufficiently short IR laser pulses for delivery and fluid delivery probe assembly that includes a hollow optical fiber and a distal sapphire tip with lateral irrigation channels.

FIG. 14A illustrates an example system for PIRL-based local tissue disruption, drug delivery, and photodynamic activation.

FIGS. 14B, 14C, 14D, 14E, 14F and 14G illustrate the use of laser assisted injection of photodynamic drugs, where the drug is designed for faster uptake than diffusion out of the tumor boundaries and the drug is activated by an additional light source resonant with photolabile groups attached to the drug molecules to create a photoadhesive which will be bound in position while creating singlet oxygen for destruction of cancer cells.

FIG. 15 illustrates the use of the optical fiber for photo fixing of drugs after local drug delivery.

FIG. 16 illustrates the use of the optical fiber to deliver additional PIRL pulses after local drug delivery to perform photomechanical-enhanced drug diffusion.

FIG. 17 illustrates the use of the optical fiber to perform photothermal treatment of the tissue region.

FIG. 18 illustrates extended-duration photodynamic therapy via an optical fiber that remains inserted in the patient after initial treatment.

FIG. 19 illustrates the use of an optical pressure sensor for the detection of tumoral interstitial fluid pressure (IFP).

FIG. 20 illustrates an example system for performing PIRL-based local tumor disruption and direct drug injection using a laser pulse delivery tool having a distal optical waveguide for beam delivery and an integrated fluidic channel for targeted drug delivery.

FIG. 21 shows ultrasound images demonstrating the ability to localize to tip of an optical fiber emitting PIRL pulses via the detection of photoacoustic signals.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As described above, cancer drugs are typically designed for systemic or full body exposure to kill the primary cancer and any migratory cells that might lead to metastases of the cancer. This puts enormous constraints on drug design to be highly specific for rapidly dividing cancer cells in which the cancer mass can be >10⁴ smaller than rest of the body mass. This high contrast is needed to try to selectively kill cancer cells over healthy cells, however, the required contrast is not perfect, which often leads to the acute and debilitating side effects associated with chemotherapy. New classes of drugs can be designed for rapid uptake rather than high selectivity to a given cancer profile. The action volume of the drug could be determined by the diffusivity of the drug into a given tissue.

It would therefore be desirable to be able to directly and locally inject a drug to tumor to provide targeted therapy and avoid the complications and limitations of conventional chemotherapy.

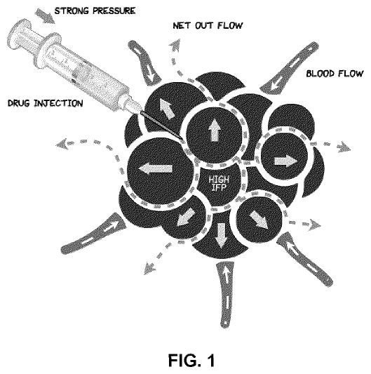

Given a well-defined cancer location, the success of direct injection with the use of a needle of a cancer drug at the site of the tumor is limited by the high osmotic pressures associated with cancerous tissue, which leads to a net outflow of the drug rather than enabling site selective treatment of just the cancer tissue, as shown in FIG. 1. In addition, the uptake of the drug is further complicated by out-diffusion away from the cancer site. There is no conventional means to arrest the diffusive loss of drug at the targeted site. The pressure gradient and associated net diffusion process leads to a loss in local concentration of the drug at the cancer site. There is a competition between drug uptake (hours) and diffusion away (seconds to minutes) from the cancer site. It is these two processes that lead to loss of efficacy in uptake of the drug in using any conventional means to deliver cancer drugs at an injection site. The same problem exists for treatment of most intracavity, localized, diseases.

The problem is exacerbated by the occurrence of chemo-immunity where, even a powerful drug that works selectively for a given type of cancer, can further influence the evolution of the cancer tissue morphology and lead to increased osmotic pressure and abnormal vasculature that can further exasperate the problem, which leads to a form of immunity from the drug action, as mentioned above. Accordingly, the direct delivery of drugs by use of needles has failed as a consequence of the enormous interstitial fluid pressure (IFP) that prevents physical delivery of the required dose to the cancer site.

These effects limit the drug dose from reaching the critical concentration required (LD50 for cancer cells) to perform the intended function. If a drug were provided in a uniform distribution, the drug would be selectively taken up by the targeted cancer site. However, the spatial gradient in diffusion at the cancer site blocks this condition. This effect occurs frequently and arises from the high degree of vascular abnormality and buildup a high osmotic pressure with further changes in tissue morphology induced by the drug action itself. Indeed, in some solid tumors the osmotic pressure becomes so high that it is physically not possible to inject the drug with normal needle stock and pressure delivery. It is simply a matter that it is not possible to get the drug to the cancer location due to this enormous pressure gradient inherent to cancerous tissues. As noted above, the subsequent problem is normal diffusive dispersion of the drug from the site even when site-selective delivery is possible. The present disclosure provides solutions to this diffusion problem in both the regimes of normal and cancerous tissue.

Another problem with direct injection of drugs into solid tumors is access to the tumor location. Large open wound surgeries cause significant trauma and are a limiting factor in the number and frequency of treatment of solid tumors in multiple different locations. The use of surgical intervention to remove cancerous tissue is limited in scope due to the significant trauma to surrounding tissue and loss of function. Usually only one or few surgical procedures can be tried to eradicate cancer. Metastasis to multiple sites leads to stage 4 cancers that no longer can be removed surgically to extend life. Less invasive procedures can be attempted to minimize the damaged volume of tissue such as needle aspiration or related delivery methods.

However, a hollow needle mechanically pierced into the body can cause trauma through the shear force needed to push the needle to the targeted tissues site, limiting the ability repeat treatment. In addition, numerous flexible guided needles have been developed to reach targets inside the body via curved entrance paths which also lead to damage to tissue along the entrance wound (e.g., Van de Berg, Nick J.; van Gerwen, Dennis J.; Dankelman, Jenny; van den Dobbelsteen, John J. (2014). Design Choices in Needle Steering&#x2014; A Review. IEEE/ASME Transactions on Mechatronics, ( ), 1-12.doi:10.1109/TMECH.2014.2365999).

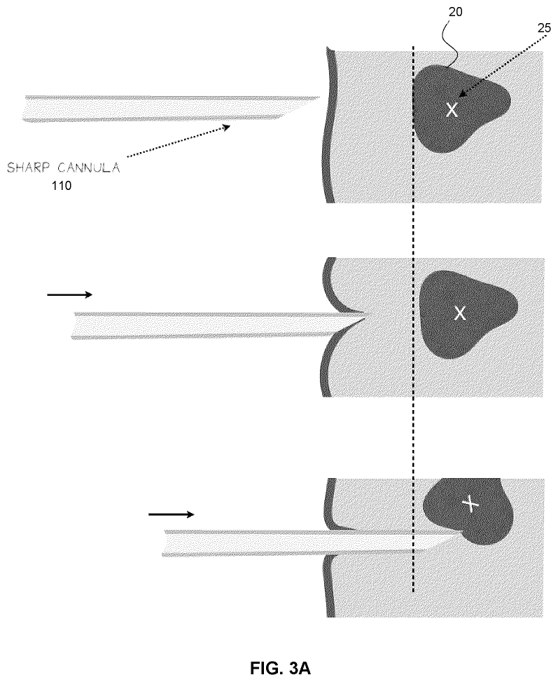

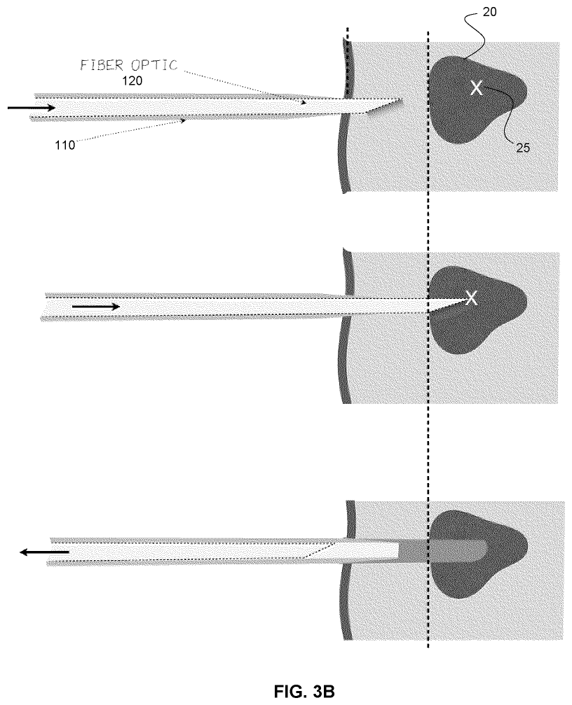

FIG. 3A illustrates the challenges faced when attempting to penetrate tumor tissue 20 and reach a target location 25 using a conventional cannula 110 having beveled tip (e.g. a needle). As the needle 110 is advanced to initially contact the skin surface, the surface deforms, and as the needle breaks through the tissue surface, the surface is locally depressed (dimpled). Eventually the elastic limit is reached and the tissue spreads to accommodate the needle. As the needle continues to advance into the tissue, the surrounding tissue deforms further. The cells are displaced or pushed apart as the needle advances towards the tumor and cuts a path through the tissue. The needle compresses and deforms tissue, enroute to the desired location within the tumor. The needle causes damage to tissue by the both the shear force trauma of the needle entry and the deformation of tissue, which causes difficulty in reaching the desired region, especially in the presence of significant tissue heterogeneity.

When the needle contacts the tumor, it does so at a location that is offset from a planned location due to deformation of the tumor caused by advancement of the needle and deformation of the tissue surrounding the tumor. Further advancement of the needle causes further deformation of the tumor, as the high interstitial fluid pressure of the tumor resists penetration of the tumor by the needle, and the distal tip of the cannula 110 fails to reach the target location 25 within the tumor 20.

The present inventors, when setting out to solve the aforementioned problems associated with local delivery of a therapy to a tumor site, identified the following challenges: 1) the need to guide an optical fiber to tumor tissue without inducing substantial tissue deformation, to enable more accurate targeting of the tumor, 2) the ability to penetrate a tumor and overcome the interstitial fluid pressure to facilitate the delivery of the necessary volume of the drug, delivered either in a solution or gas; and 3) the need to limit diffusion of the drug away from the cancer site once delivered, in order to enable drug uptake. The need to block diffusion away from the cancer was deemed by the inventors to require a direct intervention to change the flow gradients responsible for the high interstitial fluid pressure in cancerous tissue. The present inventors therefore sought a solution that would facilitate the local delivery of therapy targeted at an intracorporeal tissue region (e.g. a tumor or other region of tissue pathology), preferably while only substantially affecting the intracorporeal tissue region, without causing significant trauma or deformation to neighbouring tissue, thereby potentially enabling the procedure to be repeated multiple times without compromising quality of life.

As explained in detail below, the first and second challenges may be overcome with the use of a pulsed (e.g. ps or ns) infrared laser that delivers pulses having properties that result in local disruption and liquification of the tissue, enabling penetration of tumor tissue and the creation of pathways inside the tumor that are much smaller than would be possible with mechanical entry injection needle, and without substantial deformation of tissue as typically associated with needle-based biopsies.

This approach provides access to the tumor site with substantially less trauma than conventional approaches and enables selective control of energy deposition solely at the tumor site, which may be beneficial in reducing the elevated pressure of the tumor and facilitating the subsequent local dispensing of a therapeutic agent within the tumor.

Accordingly, various example embodiments of the present disclosure provide systems and methods that advantageously employ optical-based local tissue disruption and liquification to facilitate the direct delivery of local therapy to an intracorporeal tissue region (a tissue region within the body). As will be described in detail below, local tissue disruption and liquification can be achieved using a pulsed infrared laser that is configured to deliver pulses that selectively target vibrational absorption in the tissue and are delivered with a suitable pulse duration and fluence. For example, the wavelength of the infrared laser pulses can be selected to target vibrational absorption of water to create highly localized tissue disruption due to the extremely strong absorption of infrared in the OH-stretching region, with absorption 1/e depths on the order of 1-10 microns, which is smaller than a single cell dimension.

A pulsed infrared laser system that is configured for the delivery of laser pulses having pulse conditions suitable for performing tissue disruption according to the aforementioned mechanism and the conditions described in further detail below is henceforth referred to as a “PIRL” (pulsed infrared laser) system. Likewise, infrared laser pulses having wavelengths, pulse durations and energies suitable for performing tissue disruption and liquification according to the aforementioned mechanism are henceforth referred to as a “PIRL” pulses. It will be understood that a PIRL pulse is not limited to a picosecond pulse, as preferred pulse durations for some wavelengths extend into the tens of nanosecond range, as described below.

PIRL laser pulses are infrared laser pulses that are sufficiently short to drive tissue disruption and liquification faster than the timescales associated with thermal and acoustic transport, thus avoiding damage due to heat and shock wave formation, while also being sufficiently long to avoid the ionizing radiation effects of plasma formation. PIRL pulses are provided with a wavelength selected such that absorption of the laser pulses by tissue is predominantly due to excitation of vibrational modes of one or more constituents of the tissue, such as water. Example suitable wavelength ranges for PIRL laser pulses therefore include 2.7-3.3 μm, 5.9-6.1 μm and 1.8-2.0 μm. Future developments in high energy and short pulsed laser sources will enable PIRL tissue disruption and liquification by targeting vibrational absorption in target molecules between 2-20 μm.

For example, the PIRL laser pulse wavelength may be selected to overlap with, or reside proximal to, a strong peak in the vibrational spectrum of a constituent of the tissue, such as the CC-stretch region of collagen or N—H stretch of amino acids in proteins, where there is less water for energizing materials. Such vibrational modes quickly absorb the electromagnetic radiation and may effectively localize optical energy to micron scale deep sections of the exposed tissue. In the case of water, maximum absorption for vibrational modes occurs between about 2.7-3.33 μm, where broad peaks, >10 cm⁻¹, in the absorption spectrum, correspond to the short lived, subpicosecond to picosecond, relaxation of the OH-stretching vibrational modes of liquid water molecules to energize the surroundings. The spectrum also shows the resonance conditions between the OH-stretch and other vibrational modes such as the OH bend and intermolecular modes. Other absorption peaks, for example, at approximately 1.9 μm or approximately 6 μm, may alternatively be employed, as described in further detail below.

In various example embodiments, PIRL pulses are generated and delivered such that when a given volume of tissue is irradiated, the pulse duration is shorter than (i) the time duration required for thermal diffusion out of the laser-irradiated volume of tissue, and (ii) the time duration required for a thermally driven expansion of the laser-irradiated volume of tissue. The skilled artisan will be able to determine a suitable pulse duration for PIRL pulses for a given pulse wavelength and absorption depth in tissue (e.g., in a given type of tissue). In general, for a given PIRL laser pulse wavelength that is selected according to the aforementioned criterion (absorption of the laser pulses by tissue is predominantly due to excitation of vibrational modes of one or more constituents of the tissue), the known properties of the tissue, such as the absorption depth of the laser pulses, thermal diffusion constant, and the speed of sound, may be employed to calculate a suitable PIRL pulse duration that satisfies criteria (i) and (ii) above. Alternatively, or additionally, experiments may be performed to determine a suitable laser pulse duration that satisfies criteria (i) and (ii).

For example, in the case of disruption and liquifying tissue with a laser wavelength of 3 μm, for which the absorption depth is approximately 1 μm, the maximum pulse duration can be calculated based on the ratio of absorption depth to speed of sound, 1730 m/sec, i.e. t=a/v=10⁻⁶ m/1.730×103 m/s=5.78×10⁻¹⁰ sec, giving approximately 600 ps (e.g. see Duck, F. A., Physical Properties of Tissue, Academic Press, London, 1990, and Duck, F. A., Propagation of Sound Through Tissue, in “The Safe Use of Ultrasound in Medical Diagnosis”, ter Haar G and Duck, F. A, Eds., British Institute of Radiology, London, 2000, pp. 4-15).

Different tissue types (e.g., bone, brain and skin) will have different absorption depths at a given wavelength. Around the OH-stretching band, the absorption of the tissue is dominated by the water content. In general, the absorption depth will be longer than pure water. At a wavelength of 2.95 μm, the absorption depth of pure water is close to 0.7 μm, and given the variance in the high concentration of water in different tissues, along with other OH-stretching modes in the tissue, the absorption depth may be approximately 1-2 μm at this wavelength. If the wavelength of the laser is shifted, e.g., to a wavelength of 2.75 μm, then the absorption depth of the light increases by a factor of about 3 according to the change in the absorption spectrum of the OH-stretch. (See, for example, Diaci, J., J. Laser and Health Acad. 2012, 1-13 (2012).

In another example in which tissue is disrupted and liquified using a laser wavelength of 6 μm, for which the absorption depth is approximately 100 μm, the pulse duration should be chosen as shorter than 100 μm/1.753×103=57 ns. Likewise, an absorption depth of 100 μm is expected to occur for a laser wavelength of 1940 nm. Accordingly, a suitable pulse duration for PIRL pulses will depend on the pulse wavelength. In some example implementations, a suitable pulse duration for PIRL pulses may range from 100 ps to 100 ns, depending on the selected wavelength and light intensity dependent changes in absorption depths due to saturation of the absorption at a given wavelength.

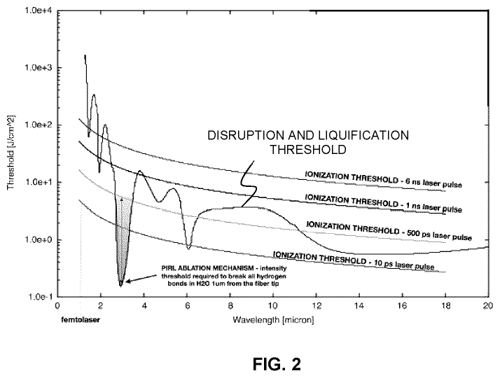

The pulse duration and pulse fluence are also selected such that a peak pulse intensity is below a threshold for ionization-driven ablation to occur within the laser-irradiated volume of tissue. For example, for a given pulse duration, a suitable upper limit of the pulse fluence may be determined to avoid the threshold for ionization-driven ablation. In the example case of human skin tissue, at a laser wavelength 3 μm, the maximum fluence values for avoiding ionization-driven ablation, for pulse durations of 10 ps, 500 ps, and 1 ns, are approximately 1.5 J/cm², 5.5 J/cm², and 17 J/cm², respectively, as shown in the FIG. 2.

Furthermore, in order to achieve PIRL-based disruption and liquification of tissue for laser pulses that satisfy the preceding criteria involving wavelength, pulse duration and pulse fluence, the laser pulses should be provided with a sufficient pulse fluence to achieve a threshold energy density for PIRL tissue disruption and liquification, as shown, for example, by the tissue disruption and liquification threshold identified in FIG. 2. For example, the pulse fluence that is delivered to the tissue should be sufficiently high so that the energy deposited in the irradiated volume is sufficient to heat the contents of the volume up to its vaporization temperature including the enthalpy of vaporization.

For example, if the beam is focused to 200 μm (or a 200 μm core diameter fiber is used in contact) and ablates a volume of ˜1 μm deep×π(100 μm)², the mass of the ablated volume is 3.1×10⁻⁸ g in the case of water and 3.4×10⁻⁸ g in the case of skin (which has a density of 1.15 g/cm³). The energy required to raise the temperature of this volume of water from 20 to 100° C. and then vaporize the volume is approximately 80 μJ of energy, which corresponds to a fluence of 0.25 J/cm² for a 200 μm spot. This fluence defines the threshold for impulsive heat deposition to drive the phase transition without loss due to acoustic transport or thermal diffusion out of the excited zone. To ensure the ensuing tissue disruption and liquification process occurs in this limit, for highly scattering medium such as tissue which effectively decreases the incident intensity, typical excitation conditions used are 1 J/cm². The determination of a sufficient fluence for PIRL tissue disruption and liquification can be made experimentally by varying the applied fluence, examining the resulting tissue disruption and liquification, and selecting an applied fluence value that provides a sufficient amount or degree of tissue disruption and liquification. The subsequent process in the confined volume, defined by the unexcited tissue and the fiber tip, leads to disruption of the tissue to cellular levels and removal of pressure gradients.

In the present implementation of contact mode delivery of PIRL pulses, localized tissue disruption/liquification occurs and allows the optical fiber to advance without substantial shear friction (analogous to a hot knife in butter), leading to fine tissue disruption, tissue homogenization, and removal of vasculature and interstitial fluid channels. In the present direct-contact implementations, the optical fiber closes the space to confine the energy. Unlike ablative PIRL that employs a non-contact mode to facilitate ablative vaporization, contact-mode PIRL does not result in transduction of the energy into translational motion as an ablation plume with material removal, but rather leads to homogeneous nucleation involved in phase transitions whereby the energy surpasses the barrier to liquify solid material and form gas bubbles from lowest vapour pressure constituents. Without intending to be limited by theory, it is believed by the inventors that the nucleation process and bubble formation occurs in a spatially uniform manner, corresponding to the energy distribution deposited in the tissue by the fast conversion of absorbed infrared radiation into thermal motions. These nucleation sites merge at longer times (>10-100 ns) and create shock waves, and this process is highly localized through the initial uniform homogeneous nucleation that occurs with the PIRL process. Ultrasound imaging has shown these uniformly generated nucleation sites to be very effective in deliberately disrupting tissue with a very fine dispersion of tissue. This process effectively liquefies tissue. Moreover, this process is cumulative locally. One can deliberately control the number of pulses to drive additional energy into the exposed volume to achieve thermal disruption which disperses to increase the volume of tissue disruption. Accordingly, in some example implementations, trains of pulses of varying lengths can be employed to increase the volume of tissue disrupted in a controlled manner to extend the tissue disruption beyond the single pulse limit of a few 10s of microns and to control the degree of tissue heating as desired.

When PIRL pulses are delivered to a local tissue region within the body, through an optical fiber inserted into the body, the resulting local tissue disruption and tissue liquification enables atraumatic and accurate guidance of the distal tip of the optical fiber to an intracorporeal tissue region of interest, and facilitates direct entry of the distal tip of the optical fiber into a selected intracorporeal tissue region, such as a tumor or other region associated with pathology.

These beneficial properties of an optical fiber delivering PIRL pulses is illustrated in FIG. 3B, in which the cannula 110 is shown housing an optical fiber 120 that is interfaced with a pulsed infrared laser source and delivers PIRL pulses from its distal tip 122. The PIRL pulses disrupt the local cellular structure and cause local tissue disruption and liquification in the laser irradiated tissue volume that lies beyond the distal tip of the optical fiber, enabling the advancement of the optical fiber along a substantially straight path, without applying a deformation-inducing force to the surrounding tissue.

As shown in the figure, the initial penetration of the tissue is facilitated by the delivery of PIRL pulses by the optical fiber, which causes local tissue disruption and liquification as the distal tip passes directing into the tissue, without substantial resistance, and without dimpling and deformation of the tissue surface. Accordingly, the fiber punctures the tissue surface with significantly less force and friction than in the conventional approach shown in FIG. 3A. The disrupted cells in the form of a liquid squeeze out behind the distal portion of the optical fiber. While the figure illustrates an example case in which the optical fiber is extended from the distal end of the cannula during PIRL-mediated penetration, insertion and translation into the tissue, in an alternative implementation, the distal tip of the optical fiber may reside adjacent to the distal end of the cannula 110 during PIRL-mediated penetration, insertion and translation, with the PIRL pulses causing tissue liquification in the direct path of the distal end of the cannula.

As shown in FIG. 3B, at least a proximal portion of the optical fiber may be housed within a cannula to mechanically support the optical fiber as it is guided within the body. When a cannula is employed during guidance of the optical fiber within the body, the cannula preferably has an outer diameter, at least within a distal region of the cannula, that only exceeds that diameter of the optical fiber by a small margin, such as less than 5%, 10%, 15%, 20%, or 25% of the diameter of the optical fiber, in order to ensure that the additional thickness of the cannula does not result in substantial tissue trauma as the cannula is advanced in the body. In some example embodiments, the diameter of the cannula tapers down in a distal direction toward the distal aperture to reduce friction and allowing the laser to ‘clear’ the path, while the cannula stretches its way into the tissue with minimal drag.

As shown in FIG. 3B, the tissue disruption and imparted fluid flow properties allows the fiber to advance to the tumor with little or no discernable friction or shear sticking force. This property enables clear trajectory paths to the tumor site without substantial tissue deformation and ensures arriving at the desired targeted tissue or region of interest. As will be described in further detail below, this property of fiber-delivered infrared laser pulses also enables the injection of therapeutic agents without substantial damage to the tissue along the entry pathway to the target site and ensures arrival at the target site without substantial tissue deformation.

FIG. 3B also shows how the disruption and liquification of tissue by the PIRL pulses emitted by the distal tip of the optical fiber enables the distal tip of the optical fiber to enter the tumor 20 without incurring a significant resistance and without being significantly deflected. As a consequence, the PIRL pulses emitted by the optical fiber facilitate the direct and unimpeded entry of the optical fiber into the tumor 20, in the absence of substantial deformation of the tumor, enabling the positioning of the distal tip of the optical fiber at the desired intratumoral location 25.

The PIRL pulses delivered within the tumor reduce the barriers to free flow of interstitial fluid into hypoxic portions of the solid portion of the tumor. The local disruption of the tumor tissue that is caused by the infrared laser pulses is similar in concept to the local high-energy radiation therapy (e.g., the “gamma knife” or “cyberknife” technologies) that employ concentrated gamma or high-energy x-ray radiation to create local spurs of ionization at select sites. Such systems require major instrumentation/cost in the form of a particle accelerator to obtain the desired radiation, and such methods operate on a probabilistic model for spur formation. The systems and methods of present disclosure, in contrast, can be implemented using a compact, comparatively low-cost, table-top laser with an optical fiber for delivery of the laser energy in a form that effectively creates its own “tunnel” to the specific target site, where the target site is known from preoperative imaging.

Some example embodiments of the present disclosure employ a thin rigid or flexible cannula to facilitate insertion and guidance of the optical fiber into the body. In other example embodiments, a fiber may be employed, in the absence of a supporting cannula, to access and provide local therapy to an intracorporeal tissue region, with atraumatic fiber delivery, and accurate fiber positioning, facilitated by the emission of PIRL pulses and the resulting disruption and liquification of tissue residing beyond the distal tip of the optical fiber. Indeed, in cases in which the optical fiber is guided in the absence of cannula, the wound size is only slightly larger than the fiber diameter, which may be, for example, in the range of 50-200 microns in diameter. Due to the small size of the disruption filament and the non-thermal disruption mechanism, minimal damage is caused to the tissue along the path towards the final target within the body.

In some example implementations, an optical fiber may be inserted, without mechanical support by a cannula (e.g., extended from a cannula, or inserted without a cannula), approximately 5 cm into the body without substantial risk of breakage for soft tissue. An optical fiber may be extended up to this depth by control of the advancement of the optical fiber for example, at a low laser repetition rate, such as 10-100 Hz, to allow the optical fiber to advance without thermal accumulation to cause damage. It is expected that more rigid tissue structures will deform around the optical fiber, but if the fiber movement is too fast there will be resistance that may break the fiber. With 5 cm penetration, most positions in the body can be accessed. It is expected that this depth can be increased to 10 cm or more, providing full access within the body, using a cannula or line-of-sight laser drilling deeper into the tissue with aspiration of the disrupted/liquified tissue.

For structures that are more rigid, such as collagen or ligaments that are in the way of the path to the tissue of interest, another laser could be used to create a small access hole (with the laser energy from the other laser being coupled into and delivered through the same optical fiber). Examples of suitable lasers include an excimer or 266 nm Nd based laser, which can be employed to create the pathway. In such a case, it may be beneficial to include a means of removing the disrupted tissue to reduce the buildup of retarding force and chance of inhomogeneous stress leading to fiber breakage.

For example implementations that employ a cannula for fiber support, the cannula (or other support structure) could be positioned to a depth that is close to the surface of the body, or into the body to a location below the surface where damage is minimal. The optical fiber can then be carefully advanced, withdrawn, material aspirated to reduce back retarding force, and the optical fiber may then be advanced further beyond cleaned out point. This procedure could be performed intermittently during advancement of the optical fiber, for example, every 5 mm of advancement (it will be understood that the distance between aspiration events will be tissue specific). In many cases, it is expected that the local disruption and liquification of tissue by the PIRL pulses will provide sufficient liquification of the tissue to permit the liquified tissue to pass longitudinally along the fiber or cannula outer surface as the fiber is advanced into the body.

In some example implementations, one or more optical fibers (e.g., an optical fiber bundle) may be employed to facilitate an endoscopic pulsed infrared laser beam delivery device having distal transverse cross-sectional dimensions less than one millimeter, unlike previous endoscopic approaches which necessarily involve dimensions well exceeding 1 mm. For example, in some example implementations, as described in further detail below, the distal region of the probe may have a diameter less than 300 microns or even less than 200 microns for beam delivery for minimal damage to the tissue along the entry pathway. The use of an optical fiber is beneficial for the delivery of infrared laser pulses both for creating the minimally invasive path to the target tissue and for eradication of the targeted tissue.

Various embodiments of the present disclosure may thus be employed to solve the problem of cancer removal without causing substantial trauma, thereby enabling removal of cancer that can be detected within the body, even in cases of where the cancer has metastasized. Conventional treatment of cancer often stops at stage 4 where metastases has occurred as surgical intervention is no longer an option due to excessive risk in current invasive surgical procedures. The embodiments of the present disclosure provide a novel means of energy delivery without any shear damage to surrounding tissue to gain access to region of interest. Multiple surgical procedures can be executed to remove multiple cancers without trauma even for stage 4 patients and for greater efficacy of drug delivery.

With fiber delivery of PIRL pules, the entry level wound is on the order of 10-20 cells in diameter and without shear induced damage, elastic rebounding of tissue occurs to close the wound, which can be affected without substantial damage along the entry path to the targeted tissues to be removed. In contrast, in the case of a needle, there are shear forces in the insertion process that lead to inflammation around the entry path and it is this effect that is responsible for some of the action and benefits attributed to acupuncture. Further, the very action of the shear forces involved and differential stickiness, surface adhesion, or variations in tissue stiffness leads to deflections of needles from the target site making it difficult to place the needle on target and often requires multiple attempts to statistically improve the chance of docking the needle at the desired site.

The embodiments of the present disclosure facilitate the insertion of an optical-fiber-based probe having sub-millimeter distal cross-sectional dimensions, significantly smaller in diameter than acupuncture needles. By the action of the PIRL guided in the optical fiber and the resulting tissue interaction, the act of optical fiber entry creates its own path, without significant collateral damage or excessive shear force, to go directly to the desired site without tissued deformation or deflection of path, leading to wound-free entry into any part of the body along a preferred or surgically essential pathway. The process can be likened to a “hot knife going through butter” but with minimal (in some cases, effectively zero) restructuring of local tissue (butter in this analogy) or changes due to shear forces. The resulting wound heals without scar tissue formation to give the absolute minimum trauma to the body to allow removal of diseased tissue. This feature enables multiple surgeries without risk or undo trauma to the patient for all procedures with especially important applications for removing solid tumors for patients to give an extended and higher quality of life.

Current approaches to minimally invasive surgical interventions, such as manual/robotic endoscopic or laparoscopic surgery, can be performed using surgical navigation (guidance) methods, but the cross-sectional dimensions of such devices are still relatively large, with trocar sizes typically in the range of 8.5 mm to 12 mm. These methods of intrusion necessarily introduce damage, especially in entering soft tissue, where shear forces lead to local damage and inflammation, even If healing of the entry wound is not debilitating.

The path of the fiber to this site can be tracked in real time with minimal tissue deformation during passage to ensure absolute targeting of the desired tissue, or precise location. In some example implementations, a probe assembly that includes an optical fiber and a cannula (or in some cases, an optical fiber absent of a supporting cannula), can be guided and adjusted in real time with one or more imaging methods such as, but not limited to, ultrasound imaging, magnetic resonance imaging, fluoroscopy, computed tomography, angioscopy and electromagnetic position sensing, optionally employing one or more detectable markers residing on the cannula and/or optical fiber, and optionally also providing surgical guidance based on intraoperative volumetric image data that is rendered in an intraoperative frame of reference and presented on a user interface.

In some example embodiments, the optical fiber tip may be detected and localized by ultrasound imaging of a photoacoustic signal generated by the local disruption of tissue by the PIRL pulses. For example, ultrasound imaging may be employed to detect shock waves that enable localization of the distal tip of the optical fiber. Such an embodiment may be beneficial in providing an improvement of localization accuracy over what is achievable based on the detection of ultrasound echoes from the tip region of a sapphire optical fiber, as ultrasound artifacts can arise due to the high reflectance due to the acoustic property difference of 10 (v_sapphire=10× speed of sound in water and >10× for speed of sound in tissue), as a high reflectance to low reflectance boundary leads to constructive interference that can impair visualization of the precise location of the tip. By superimposing ultrasound imaging with photoacoustic imaging, the active region at the distal end region of the fiber can be imaged to high accuracy. If ultrasound imaging is employed with a sufficiently high frequency, it can be possible in some cases to also image cancerous tissue due to increased vascularization within a tumor region. For example, the projection the acoustic signals generated by PIRL tissue disruption onto acoustically imaged tumor tissue, as highlighted by phase contrast and ultrasound image contrast agents (bubbles or nanoparticles), can provide intraoperative guidance for facilitating accurate positioning of the fiber and the appropriate delivery of therapy to the tissue of interest.

The use of ultrasound imaging is limited in depth resolution by acoustic attenuation that varies quadratically with frequency. To have sufficient spatial resolution to image the fiber position, ultrasound wavelengths of approximately 30 MHz (or approximately 30 micron acoustic wavelengths) may be beneficial in enabling near diffraction limited resolution with current transducer technology. This frequency limits the imaging depth to approximately 1-2 cm. This depth can be extended to approximately 5 cm by employing lower frequencies, such as frequencies between 10-20 MHz.

In order to provide accurate position tracking and guidance deeper within the body cavity, other imaging modalities that have less contrast may be employed to identify the position of the fiber in the 3D space of the body cavity. For example, fluoroscope x-ray imaging or use of electromagnetic field gradients or MRI may be employed to image the fiber and guide its position. Such imaging modalities may not have sufficient contrast to identify the fiber position relative to critical components within the body, to guide the fiber along the optimal, surgically relevant, pathway.

In such cases, as well as a general feature of imaging the fiber within the body, the position of the distal tip can be uniquely determined by exploiting the large strain field generated by the PIRL process at the fiber tip. The action of PIRL in tissue disruption via the ultrafast thermal energy deposition in the 10 micron scale leads to thermally driven volume expansion with strain fields (delta V/V) of 10⁻² or larger. This strain field is orders of magnitude larger than that radiated by piezoelectric transducers used for the ultrasound imaging. This extremely large strain can be detected by conventional photoacoustic detection to give an extremely bright acoustic point source or beacon to uniquely position the fiber in the body. The extremely large magnitude of the strain field means that the signal can be detected up to the desired 5-10 cm depth, or deeper, that would then effectively give unique photoacoustic location of the fiber anywhere in the body. This acoustic beacon, which is inherently generated through the action of the PIRL pulses emitted by the fiber in the act of tissue disruption to make a pathway or for deliberate tissue disruption and thermally driven apoptosis, can be superimposed on other previously generated images such as CT scans, MRI, or use of position sensing devices (e.g. electrostatic location of the fiber tip). In the latter case, a pick-up coil can be used to map and display the fiber position relative to a 3D CT scan or other reference pre-operative volumetric image (provide that the preoperative image can be represented in the intraoperative reference frame, for example, by the use of stereotactic patient tracking devices, such as optical tracking systems, and/or via intraoperative image fusion, such as ultrasound to CT image fusion). The photoacoustic beacon thus provides a means of locating the fiber tip and its relation to the planned surgical route to the tissue of interest.

Once the distal end of the optical fiber is positioned within an intracorporeal tissue region of interest, such as a tumor, the optical fiber may be employed to transmit PIRL pulses with sufficient energy to disrupt a desired volume of tissue, for example, via one or more pulses, optionally with timed intervals suitable to achieve a desired volume of tissue disruption and/or killing of a specific volume of tissue. For example, after having reached the target site using a laser fluence appropriate for piercing the tissue, the energy and/or power of the infrared laser pulses may be increased to accelerate tissue disruption at the tip, and the tip can be scanned to increase the volume of disruption at the tumor site to eradicate the diseased tissue.

It will be understood that the skilled artisan may perform experiments with real or simulated tissue (e.g. a phantom) to determine a suitable pulse energy, number of pulses, and or pulse repetition rate to achieve a suitable level of PIRL-based disruption and/liquification, and/or thermal therapy induced apoptosis. For example, in the case of PIRL pulses tuned to the OH stretch water resonance at a wavelength of include 2.7-3.3 μm, each pulse interaction above the threshold for tissue disruption has been found to disrupt and liquify approximately 10-100 micron depth profile (depending on the chosen wavelength and the mechanical properties of the tissue) from the optical fiber exit face.

Once the optical fiber has penetrated a tissue region of interest via PIRL-induced tissue deformation and liquification, thermal diffusion from the fiber tip location can be exploited within a defined volume element to cause thermal-induced apoptosis. The optical fiber can be placed at a desired location in the target tissue and thermal diffusion can be exploited to deliver energy, e.g. either by continued irradiation with the PIRL laser or use of a WDM in fiber system to use any other wavelengths, such as 532 nm (green) light to deposit energy through other absorption bands (such as, for example, hemoglobin absorption bands in blood) to heat the tissue to apoptosis, providing a well-defined kill zone for cancer or eradication of other diseased tissue.

Tissue necrosis can be achieved with programmed temperature variation (delta T) within a controlled zone. Depositing known powers to heat the tissue and raise the temperature of the desired boundary, for example, up to 60° C., can be employed to kill the tissue. At this temperature, cells undergo “programmed cell death” or apoptosis. For most applications, simple diffusion models can be used to accurately determine the required power and time of exposure to lead to cell apoptosis out to a desired tissue diameter. An optical temperature sensor, of which many various designs exist, such as a fluorescence monitoring, phase interference monitoring or reflectometery including a Bragg grating written into the tip of the fiber can be incorporated in the distal end of the waveguide, which can monitor the localized temperature in-situ during the photothermal exposure to ensure proper irradiation protocols to induce apoptosis out to the desired tissue volume.

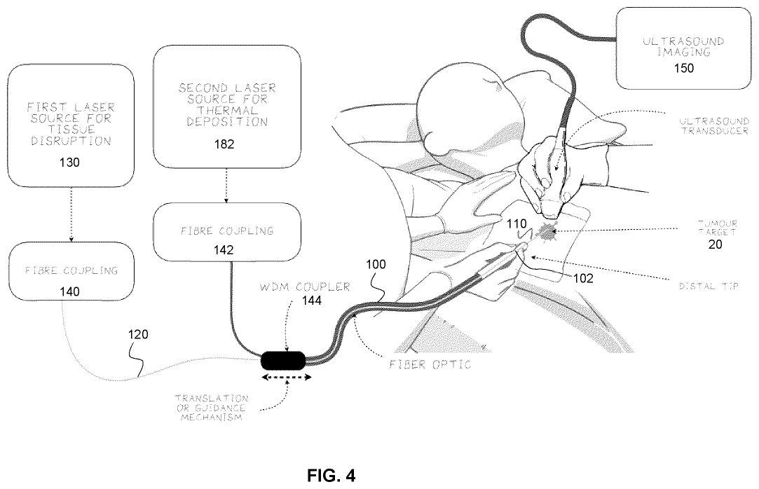

FIG. 4 illustrates an example system for PIRL-based local tissue disruption and thermal therapy. An optical fiber 120 delivers PIRL pulses from a PIRL laser system 130 to a probe body 100. PIRL pulses may be coupled from the laser source into the optical fiber 120 via coupling 140 (which may be a rotary optical joint to facilitate rotation of the optical fiber relative to the PIRL laser system 130; alternatively, an optical rotary joint may be housed within or on the probe body 100). An additional light source 182, capable of delivering laser energy suitable for intratumoral thermal therapy (thermally-mediated apoptosis), is coupled to the optical fiber 120, for example, though a fiber coupling 142 and a wavelength division multiplexing coupler 144. In some example embodiments, the optical fiber will be provided within and extended from an assembly that includes multiple sections of supporting enclosures (probe bodies and cannulas). In some cases, the fiber itself may be encapsulated within a metal or plastic cannula that sits within a larger metallic canula (a nested probe assembly). The fiber may protrude from the final section of this assembly bare for some distance depending on the fiber thickness and coatings.

As shown in the figure, the optical fiber 120 is received and supported by the probe body 100. A distal cannula 110 extends from the distal end 102 of the probe body. In some example implementations, the distal portion of the optical fiber 120 resides at or near the distal end of the cannula 110, or is extendable relative to the distal end of the cannula. In other example implementations, the distal cannula 110 supports a distal optical waveguide that is in optical communication with the distal end of the optical fiber 120. It will be understood that the optical fiber 120 may be formed from two or more segments. In some example implementations, at least a portion of the cannula 110 (such as a distal potion) is flexible.

The distal cannula 110 is aligned by the surgeon (or via robotic surgical subsystem) for angular placement and direct advancement to the target tissue 20, facilitated by PIRL-based tissue disruption and liquification that causes minimal collateral damage to neighbouring tissue along the path to the target tissue 20. Initial angular alignment may be provided by the distal cannula portion 110 with an accurately determined initial position at the body's entry point and forward trajectory for the target tissue, involving geometric location and delivery, assisted with an imaging and/or positioning subsystem (i.e., a “navigation” or “guidance” system) 150. After having employed PIRL pulses to facilitate the penetration of the tumor by the distal tip of the optical fiber, and after having positioned the distal tip of the optical fiber at a desired location, the second laser source 182 is controlled to deliver thermal therapy, optionally repositioning the distal tip of the optical fiber at different intratumoral locations during the delivery of thermal therapy.

While the example implementation illustrated in the figure demonstrates the use of ultrasound-based image guidance, it will be understood that any suitable image guidance system, tracking system, or positioning system may be employed to facilitate guidance of the probe to the target, optionally also providing surgical guidance based on intraoperative volumetric image data that is rendered in an intraoperative frame of reference and presented on a user interface.

As shown in the figure, the present system for intratumoral or intracavity direct drug injection includes the PIRL laser system 130 that is coupled to the optical fiber such that the output of the distal end of the optical fiber would have sufficient conditions (wavelength, pulse duration and intensity, as described above) to facilitate highly localized micro-disruption of tissue.

As noted above, the position of the optical fiber 120, or distal optical waveguide, or distal region of the cannula 110, may be detectable by and displayed, optionally relative to pre-operative image data, via a surgical guidance or navigation system, which can include a positioning mechanism, manual or motorized, which guides the insertion of the distal end of the apparatus into the body towards a targeted volume of tissue to be disrupted by the laser (disrupted tissue) within the boundaries of a solid tumor target. The position of the distal end of the optical fiber, distal waveguide, and/or cannula can be determined, for example, using a position sensor in conjunction with spatial imaging such as an ultrasound, x-ray or MRI.

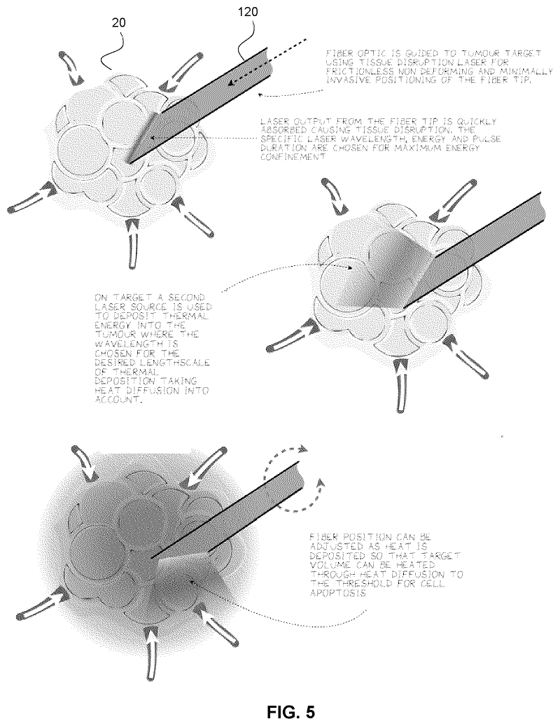

FIG. 5 illustrates the delivery of thermal therapy within a tumor 20 via the same optical fiber 120 that was employed to achieve intratumoral access via the delivery of PIRL laser pulses. After having positioned the distal tip of the optical fiber within the tumor via PIRL-mediated intratumoral access, a second laser (e.g. the laser system 182 in FIG. 4) is employed to deliver thermal therapy.

The wavelength of the second laser may be selected based on a desired length for thermal deposition, taking thermal diffusion into account. For example, near infrared 750 nm CW lasers may be employed, which absorb over approximately 5 mm deep in tissue, or green lasers such as 550 nm which absorb over approximately 0.5 mm in tissue. In some example implementations, lasers with outputs at 532 nm can be used to deposit energy into the target tissue by the absorption of light in oxygen transport pigments such as hemoglobin or myoglobin for heavily vascularized cancer tissues to have preferential absorption in the cancerous tissue and tunable wavelengths to adjust the absorption depth for energizing the target tissue to the desired temperature for apoptosis at the optimal heating rate, taking into account thermal diffusion, to more efficiently target cancerous tissue.

As shown in FIG. 5, the distal tip of the optical fiber may be repositioned and/or reoriented during the delivery of thermal therapy, for example, according to a surgical plan, to provide a desired spatial distribution and dose of thermal energy. For example, e.g. using pre-operative volumetric image data to estimate the tumor size, and known or estimated thermal conduction and diffusive transport properties of the tumor, and a known or estimated absorption depth of the second laser for a given absorption band, and a system that can use this information to determine a set of positions and orientations to ensure a requisite temperature rise, for a prescribed time interval and dose of laser exposure, to achieve apoptosis.

In some example implementations, an optical fiber delivery system delivers pulsed infrared pulses to provide a pathway for injection of liquid therapeutic agents (e.g. drugs) with minimal collateral damage to the adjacent tissue while simultaneously enable tissue disruption at the fiber exit, thereby creating pathways for drug delivery and elimination of pressure gradients common to highly vascularized cancer tissue that otherwise leads to outflow of the drug and loss of efficacy.

Some example embodiments of the present disclosure facilitate local injection of a liquid therapeutic agent (e.g. a drug) to improve effectiveness of the injected drugs, for example, for tumor destruction or other actions for medical treatments, beyond what is possible for needle stock drug delivery. For example, embodiments of the present disclosure can address the problem of chemo-immunity due to the presence of high interstitial fluid pressure, reduce the pressure by tissue disruption, and facilitate the uniform dispersal of the drug at the specific targeted site for optimal drug delivery. Some example embodiments of the present disclosure can therefore be employed to reduce the amount of drug needed to selectively attack cancer tissue, for example, by orders of magnitude, and thereby facilitate a reduction in the side effects of chemotherapy. The present methods may therefore lead to an improvement in the quality of life, potentially to near pre-cancer status, by reducing the associated side effects of chemotherapy. In some example embodiments, after drug delivery, the application of radiation or energy delivery may be employed to create barriers to physical diffusion of the drug away from the cancer site, thereby potentially further increasing efficacy.

Accordingly, in some aspects, the present disclosure facilitates the selective delivery of liquid therapeutic agents to cancerous tissue, even for high osmotic pressure tissues, and provides a means to block diffusion of the liquid therapeutic agent away from the cancer site, thereby solving the problem of uptake vs. diffusion away from the cancer, even for normal osmotic pressure conditions. Furthermore, in some aspects, the present disclosure additionally or alternatively facilitates the site-selective delivery of a liquid therapeutic agent or other material to a given location in the body without damage to the surrounding tissue in the act of the drug delivery. As noted above, some aspects of the present disclosure also enable the subsequent blocking of diffusion from the injection site to solve the problem of both high osmotic pressure that blocks entry of drugs to cancer sites and diffusion away from the site once injected. The methods of the present disclosure may avoid or reduce the dependency on systemic drug regimens by enabling access to cancer sites for site selective drug delivery. This capability can reduce the required dose, for example, by orders of magnitude, thereby avoiding side effects associated with chemotherapy.

Such embodiments of the present disclosure may be employed to avoid or reduce the need for high selectivity drugs, as is currently needed according the present standard of care treatment methods that rely on systemic treatment. Instead, for example, a drug can be delivered that is suitable for rapid uptake locally, optionally combined with means to block subsequent diffusion with the creation of barriers to diffusive transport away from the drug delivery point. The ability to directly and locally target tumor cells while they reside within the tumor may avoid the need to also target migratory cancer cells that lead to metastasis of the cancer.

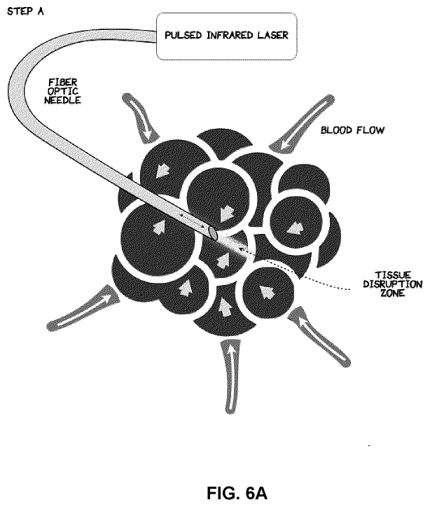

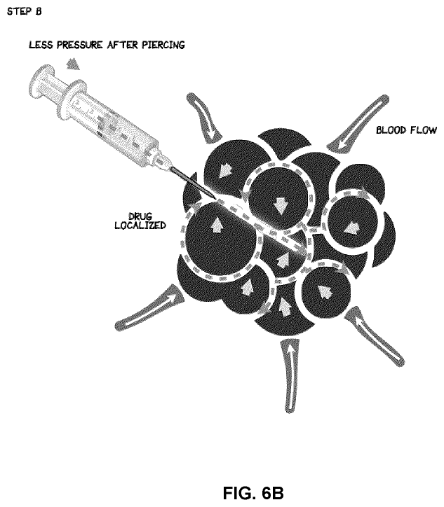

FIGS. 6A and 6B schematically illustrate the direct injection of a drug into a tumor via initial pulsed infrared laser pulse mediated tissue disruption. In step A, an optical fiber needle disrupts a zone of solid tumor tissue, thereby reducing interstitial fluid pressure by piercing/deflating. As shown in FIG. 6B (step B), a drug can be subsequently injected under lower pressure, such that the drug remains localized within the tumor with less diffusion. The process illustrated in FIGS. 6A and 6B is performed in two steps involving the initial delivery of the pulsed infrared laser pulses via first optical delivery device and the subsequent injection of drug into the optically disrupted tumor via a second drug injection device. In other example implementations, the optical disruption of the tumor and the subsequent delivery of the drug is performed using a single integrated optical and fluidic delivery device, as described in further detail below.

The third challenge noted above, namely the need to avoid diffusion of an intratumorally injected therapeutic agent, can be addressed by the selection of specialized compounds or by the additional combination of activation of light activated compounds, for example, using the same laser delivery fiber to transmit into the tumor additional light sources specifically designed for favourable drug interaction or photo-adhesion. Additional radiation can be applied at suitable wavelengths, pulse durations, and interaction times to cause localized photo-activation of a drug. The drug can be modified specifically for the purpose of light-induced fixation (photocleaving a blocking group to create reactive states that physically fix the drug to the cancer site). For example, in some example implementations, chemotherapy drugs can be employed with a means to physically fix the drug or to enhance uptake faster than diffusion away from the cancer site.

The PIRL tissue disruption and liquification mechanism avoids excessive shear force during entry of the device into tissue, thereby enabling rapid healing with greatly reduced wound entry trauma. In the case of fiber delivery, the cross-sectional wound is the size of the fiber itself, which can be on the order of 200 microns or smaller down to several tens of microns in diameter by tapering the end of the fiber, similar to the dimensions of a single cell and smaller than any acupuncture needle and where the pulsed infrared laser output from the distal tip of the instrument obviates the need for applied mechanical force. Accordingly, a fiber configured to emit infrared pulses can enter anywhere in the body with minimal trauma. As noted above, the wound size can be reduced to the diameter of approximately 10-20 cells without shear damage to the tissue, such that the tissue elastically rebounds upon exit of the fiber to leave minimal damage.

This elimination or significant reduction of trauma also means this approach can be considered for use even for metastasized cancer, as there is no or minimal additional penalty to the patient with respect to recovery. In normal cancer surgeries, large incisions are needed with massive disruption and mechanical displacement of healthy tissue to access a cancer site. According to the example methods of the present disclosure, there could be effectively no limit to the number of procedures that could be done as long as the cancer can be imaged and its spatial location determined. Such an approach renders it practical to deliberately create tissue damage by local photoactivated drug binding of a blocking agent, specific to the cancer drug, to keep the drug physically located within the cancer site by increasing the barrier to drug diffusion away from the target site, and optionally, for example, to deliver photothermal energy to achieve conditions well above thermal denaturation to the point of inducing coagulation or cauterization (with aeration).

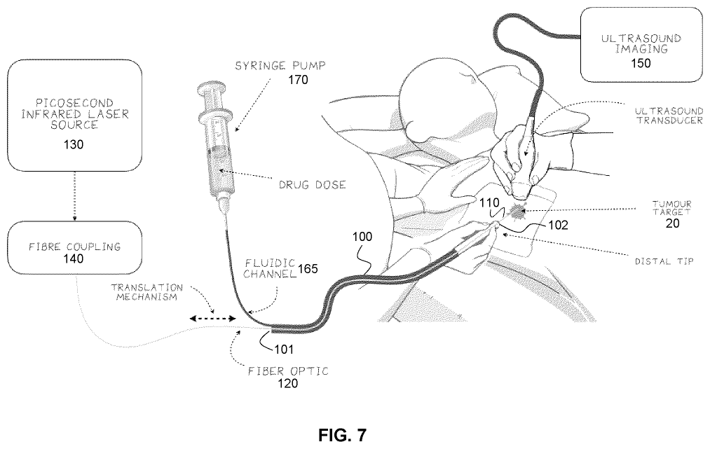

Accordingly, in some example embodiments, a probe is provided that includes an optical fiber for the delivery of infrared laser pulses and a fluid delivery conduit for the delivery of a drug. Referring now to FIG. 7, an example system is illustrated for performing PIRL-based tissue disruption of a tumor and the subsequent delivery of drug to the disrupted tumor. Similar to the example system shown in FIG. 4, the figure shows a handheld probe body 100 having a proximal end 101 and a distal end 102. An optical fiber 120 delivers PIRL pulses from a PIRL laser system 130 to a probe body 100. PIRL pulses may be coupled from the laser source into the optical fiber 120 via coupling 140 (which may be a rotary optical joint to facilitate rotation of the optical fiber relative to the PIRL laser system 130; alternatively, an optical rotary joint may be housed within or on the probe body 100).

As shown in the figure, the optical fiber 120 is received and supported by the probe body 100. A distal cannula 110 extends from the distal end 102 of the probe body. In some example implementations, the distal portion of the optical fiber 120 resides at or near the distal end of the cannula 110, or is extendable relative to the distal end of the cannula. In other example implementations, the distal cannula 110 supports a distal optical waveguide that is in optical communication with the distal end of the optical fiber 120. It will be understood that the optical fiber 120 may be formed from two or more segments, and that at least the distal portion of the cannula 110 may be flexible.

The present example system for intratumoral or intracavity direct drug injection includes the PIRL laser system 130 that is coupled to the optical fiber such that the output of the distal end of the optical fiber would have sufficient conditions (wavelength, pulse duration and intensity, as described above) to facilitate highly localized micro-disruption of tissue, substantially reducing the interstitial fluidic pressure enabling the direct and local injection of a liquid therapeutic agent. As noted above, the position of the optical fiber 120, or distal optical waveguide, or distal region of the cannula 110, may be detectable by and displayed, optionally relative to pre-operative image data, via a surgical guidance or navigation system.

FIG. 7 also illustrates an example implementation in which a fluid delivery conduit 165 delivers a drug or other fluid to the probe body 100. Fluid is supplied to the fluid delivery conduit 165 by a source of fluid (e.g., a fluid reservoir) and a pump. In the example implementation shown in FIG. 7, fluid is delivered to the fluid delivery conduit 165 by a syringe pump 170. The fluid delivery conduit 165 is interfaced with, or extends within, an internal lumen within the probe body 100, such that fluid within the fluid delivery conduit 165 is dispensable from the distal end of the cannula 110, or from a fluidic channel formed in an optical waveguide that extends from, or is extendable from, the distal end of the cannula 110. The syringe pump 170 controls the injection of a controlled dose of a pharmaceutical compound (drug) chosen for treatment of the tissue target.

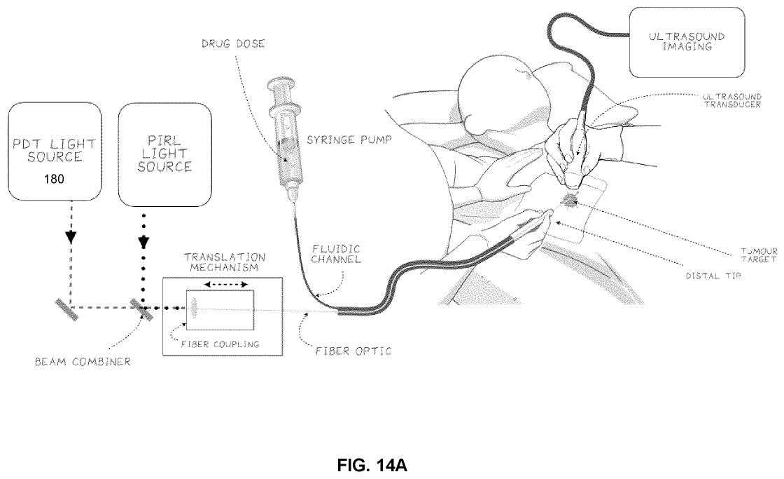

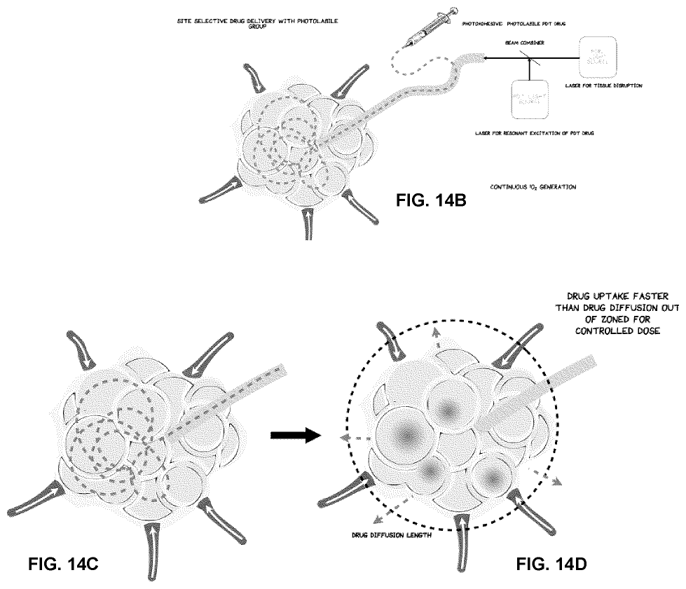

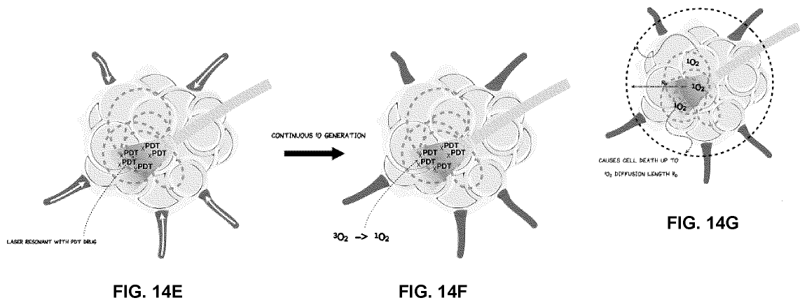

In some example embodiments, the system shown in FIG. 7 may include an additional laser source that is coupled to the optical fiber 120 and employed to deliver optical energy for additional therapy, such as, for example, to deliver local thermal therapy prior to or after having injected the therapeutic agent within the intracorporeal region of interest. An example of such an augmented system is shown in FIG. 14A and described in further detail below. For example, a dual treatment modality involving initial local thermal therapy and subsequent local delivery a therapeutic agent may be beneficial in that the local delivery of the therapeutic agent augments the deposition of laser energy to thermally kill cancer cells, further increasing the volume sampled and subject to cancer drug regimes with a high degree of selectivity to rapidly growing cancer cells and are known to conserve cell constituents to enable reabsorption of the cancer cells as typically observed as shrinkage of the tumor. The latter effect provides a dual hammer to kill cancer with higher confidence levels, and allow the cancer tissue to be reabsorbed, to lead to maximal recovery of tissue function without dead tissue blocking functions.

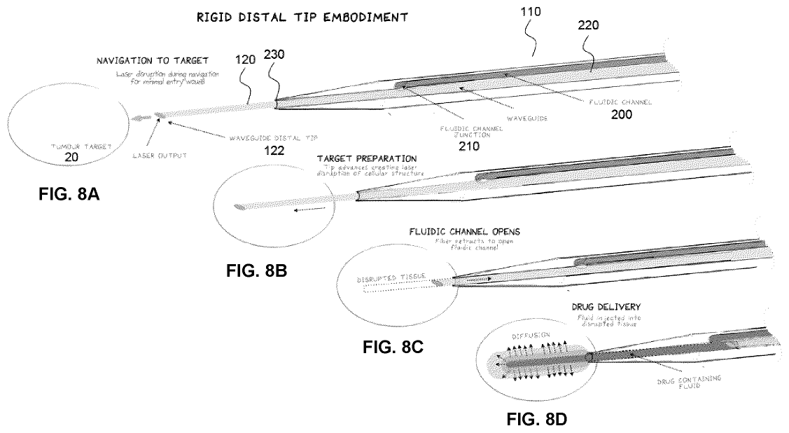

FIGS. 8A-8D illustrate an example implementation of a cannula 110 having an integrated fluidic channel (lumen) 200 and extendable optical fiber 120. At a location remote from the distal end (not shown in the figure) ports are provided for insertion of the optical fiber 120 and the fluidic coupling to the fluid delivery conduit (shown in FIG. 7). As shown in FIGS. 8A-8C, the optical fiber 120 can be advanced into the target tumor tissue 20. For example, a pullback or push-forward mechanism may be integrated into the probe body, or may reside proximal to the probe body (e.g., as shown in FIG. 14A), facilitating extension and/or retraction of the optical fiber 120. As noted above, the cannula 110 may be advanced into the tissue using a position sensing and/or guidance/navigation system, optionally while delivering PIRL pulses into the tissue during advancement of the cannula, thereby creating an entrance wound with minimal damage to surrounding tissue.

Upon arrival within the target tissue (e.g., tumor) boundary, PIRL pulses are delivered to create the intratumor disruption within the target tumor, while extending the distal end of the optical fiber 120 (or optical waveguide) into the target tissue, as shown in FIG. 8B. To facilitate intratumoral injection of the drug dose, the optical fiber 120 is retracted to a position behind a fluidic channel junction 210 residing within the cannula 110, as shown in FIG. 8C which has been primed with the drug prior to injection to avoid the injection of an air pocket. The retraction of the optical fiber 120 to a location proximal relative to the fluidic channel junction brings the fluidic channel 200 into fluid communication with the distal portion of the central lumen 220 of the cannula (through which the optical fiber 120 is extendable), thereby opening a fluidic pathway between the fluidic channel 200 and the distal port 230 of the cannula 110. Due to the disruption of the local microstructure of the tumor, the drug can be injected both into the disrupted volume of tissue and with less resistance into the interstitial fluid of the tissue due to mechanical perturbation of the extra cellular matrix, whereupon the drug can further diffuse into the tumor and overcome the previously present pressure gradients that lead to outflow of drugs from the target site, as illustrated in FIG. 8D.

While FIGS. 8A-8D illustrate an example design in which the drug is dispensed, at the distal end of the cannula 110, through the central lumen that is employed for extension of the optical fiber 120, it will be understood that other example implementations may vary in design without departing from the intended scope of the present disclosure. For example, the fluid channel may extend to a distal end or near the distal end of the cannula to a port that is adjacent to a location at which the distal end of the optical fiber resides or is extendable therefrom. In other example implementations, described in more detail below, the fluidic conduit may be a lumen residing within the optical fiber itself.

It will be understood that the distal tip of the optical fiber (optical waveguide) may be tapered or beveled. For example, FIG. 8A illustrates an optical fiber 120 having a beveled distal tip 122. While FIGS. 8A-8D illustrate an example embodiment in which a single optical fiber or optical waveguide is employed to deliver the PIRL pulses for tissue disruption, it will be understood that the waveguide may consist of a plurality of fibers (e.g., a fiber bundle). In some example implementations, at least some of the optical fibers or optical waveguides may be provided with different beveled angles, such as flat for advancing the fiber forward or include fiber cut at angles for sideways tissue disruption and increased radial removal of tissue.

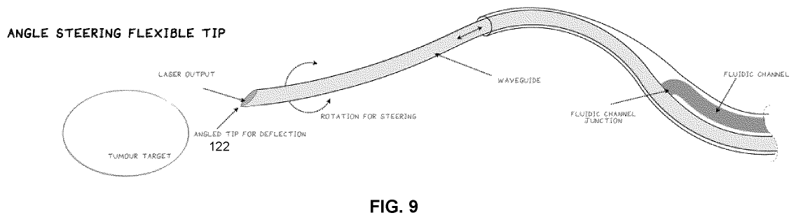

Another example of such an embodiment is shown in FIG. 9, illustrating a device in which the distal tip 122 of the waveguide is angled to create an asymmetry resulting in angular projection of the PIRL pulses. In some example implementations, the angle of deflection can be rotated about the central longitudinal axis of the fiber through rotation, where rotation is facilitated by the inclusion of a rotary fiber optical joint provided at a location remote from the distal end of the optical fiber. In some example implementations, by advancing and rotating the optical fiber, it is possible to selective destroy tissue with precision of 10 to 100 microns with the overall volume to be removed determined by the number of advancing steps and rotations.

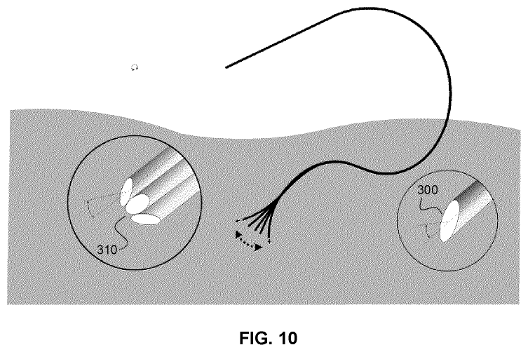

By advancing while rotating, in combination with an asymmetric distal tip of the fiber, e.g., angled like a wedge, it possible to selective guide the optical fiber along arbitrary paths with minimum curvature determined by the lateral displacement generated by the asymmetric shape and direction of laser output. An example of this angular control is illustrated in FIG. 10, which shows rotation and beam steering of a device with either a single beveled optical fiber 300 that is rotated to control the vector of advancement of the fiber either with or without assistance from the laser-tissue interaction, or alternatively a bundle of beveled optical fibers 310 in which the laser pulses can be directed into one or more fibers in sequence to control the vector of the laser-tissue interaction which would create a void of disrupted tissue into which the fiber will move preferably to other directions. Steering by conventional mechanical means using predeformed memory metals, thermally shapeable metals or opposed pulling wires can also be employed. The goal is to have the optical end face of the delivery catheter occupy the maximum amount of cross-sectional area relative to the total diameter of the distal end so as to minimize friction.

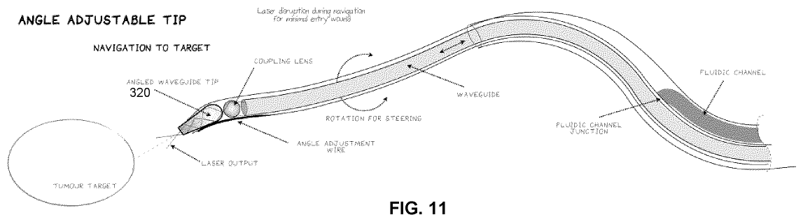

FIG. 11 illustrates another example embodiment the distal tip of the fiber includes a moveable and/or deformable distal optical component, shown in the figure as angled waveguide tip 320, in which the angle of the distal optical component 320 can be controlled remotely, e.g., via actuation of an adjustment wire or other mechanism to increase the angular displacement of the advancing fiber by mechanical deflection and/or in combination with the optomechanical effect of transmitted laser pulses.

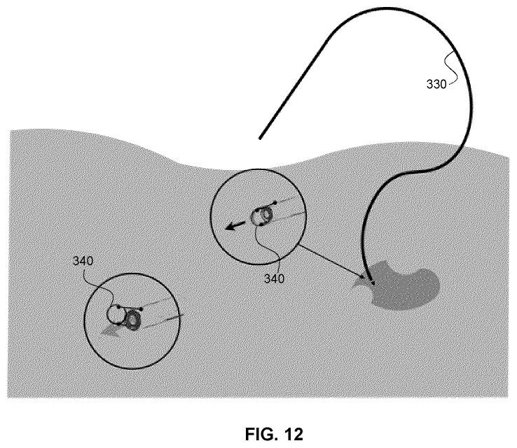

FIG. 12 illustrates an example embodiment in which the optical waveguide is a hollow fiber 330 that includes a displaceable (removable) end piece 340, such as a sapphire window/sphere/shaped tip that translates to open the irrigation channel where fluid flows inside the hollow waveguide and out the distal end of the waveguide. In some example implementations, a miniature mechanism such as a hinge or set of tension wires can be released to move the displaceable end piece 340 sufficiently far away from the tip such that fluid can be injected out of the distal end of the hollow fiber 330. The fluid flow through the distal opening of the hollow fiber 330 can be controlled, for example, by a mechanism such as, but not limited to, a piezo valve in-line with the fluidic channel.

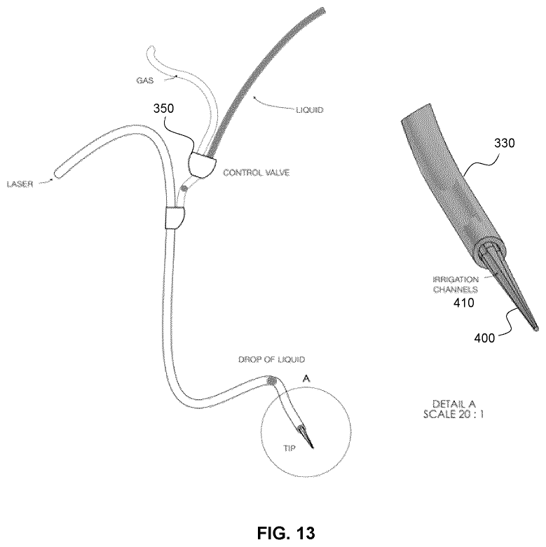

In some example embodiments, in which fluidics are coupled to a hollow fiber which share the hollow channels to the tip, gas and/or liquid can be injected into the waveguide channel to deliver liquid and then empty the channel for transmission of laser pulses, as shown in FIG. 13. Additional fluidic channels can be integrated with additional control valves for liquid or gas aspiration, irrigation, to control the pressure within the channel and for priming of the channel with liquid.

In some embodiments, multiple optical fibers (and/or cannula housing optical fibers) may be employed to treat a tissue region of interest (e.g., a tumor), with each optical fiber being inserted into the patient and directed to the tissue region of interest from a respective angle and/or insertion location. Each optical fiber may be interfaced with the PIRL laser source (optionally employing multiple PIRL sources, with each PIRL source delivering optical pulses to a respective subset of one or more optical fibers), thereby facilitating the insertion and guidance to the tissue region of interest with minimal damage, with each optical fiber acting independently such that any local damage caused while moving the fiber to the tissue region of interest does not cause aggregate damage among the adjacent fibers. In other words, the per-fiber damage can be so minimal that each fiber acts independently without any prospect for increased collective or nonlinear effects. Multiple fibers may be inserted at different angles of entry to remove nearly arbitrary volumes of tissue by the interaction of multiple shock wave fronts and/or use of the thermal energy deposition and thermal diffusion to damage tissue. The latter procedure allows even large cancers, typically comparable to golf ball dimensions, that are typically only now found with current imaging for early cancer detection.

Through the above description, this class of tumor can be thermally driven into apoptosis using moderate exposure times to laser heating the volume out to a desired diameter, followed by site selective drug delivery to kill cancer cells without risk of physical displacement of cancer cells and unintended metastases. The site selective drug delivery enables cancer eradication with orders of magnitude less dose than current drug regimes and allows for reabsorption of the cancerous tissue, observable as shrinkage of cancer volumes, to recover tissue function as much as possible.

In some example embodiments, a hollow fiber waveguide may be employed to guide the PIRL pulses for tissue disruption to create the drug delivery pathway. The hollow fiber may be equipped with a tapered sapphire tip 400 that includes one or more irrigation channels 410, as illustrated in FIG. 13. The irrigation channels may be formed laterally along the sapphire tip such that each channel is in direct fluid contact with the internal hollow region of the fiber waveguide, thereby facilitating a continuous pathway for drug delivery within the hollow fiber after optical disruption by the laser pulses. Alternatively, the irrigation channels may be connected to a separate irrigation conduit defined within the hollow fiber waveguide or within in a lumen of a sheath housing the hollow fiber waveguide. It is believed that the channels will not become blocked by the laser driven tissue disruption and/or forward translational motion of the fiber, because the action of the PIRL pulses would lead to explosive heating with force gradients that lead to material expulsion in the direction normal to the tip. This action is expected to keep the debris field from entering each irrigation channel, with subsequent PIRL pulses removing material that does enter the channels upon aspiration and acting to further advance tissue disruption for forward translation of the fiber to the target site.

As noted above, FIG. 13 illustrates an example configuration in which a sapphire end piece 400 is provided at a distal region of a hollow waveguide 330, the sapphire end piece including one or more irrigation channels 410 is in fluid communication with a central lumen of the hollow waveguide. The figure illustrates an example implementation in which a plurality of irrigation channels is located on the sides of the taper. A compact and minimally perturbative fiber delivery solution may be achieved by employing a photonic crystal or hollow fiber. The end piece, which is preferably sapphire, is IR transparent and sufficiently hard to withstand the laser driven shock waves involved in the tissue disruption subsequent to impulsive infrared pulse heating.

In example embodiments in which the fiber is a photonic crystal fiber, the sapphire end piece may include irrigation channels having proximal apertures that are in fluid communication with the hollow pores of the photonic crystal/hollow fiber. Each proximal aperture may have a diameter that is equal to, or approximately equal to (e.g., within 5%, 10%, 15%, 20% or 25%) the diameter of a respective hollow pore. The sapphire tapered end piece may include a bored hole or channel and be either fused to the hollow fiber or secured via a matching plug that only marginally increases the diameter of the fiber delivery device (e.g., an increase of less than 5%, 10%, 15%, 20% or 25%).

In some example embodiments, as illustrated in FIG. 14A, an additional light source 180 may be coupled into the optical waveguide for the purpose of photo-fixing the delivered drug compound to increase drug uptake and/or prevent rapid diffusion. In one example implementation of such an embodiment, the same distal tip shown would allow advancement of the optical fiber back to the target where the volume of the injected drug can be exposed to sufficient optical radiation for the desired photochemical outcome.

FIGS. 14B-14G illustrate the mechanism of laser assisted injection of photodynamic drugs, where the drug is designed for faster uptake than diffusion out of the tumor boundaries and the drug is activated by an additional light source resonant with photolabile groups attached to the drug molecules to create a photoadhesive which will be bound in position while creating singlet oxygen for destruction of cancer cells. This latter step allows continuous monitoring of cancer shrinkage and adaptation of light levels for controlled dosing of singlet oxygen to adjust the cancer treatment as needed.

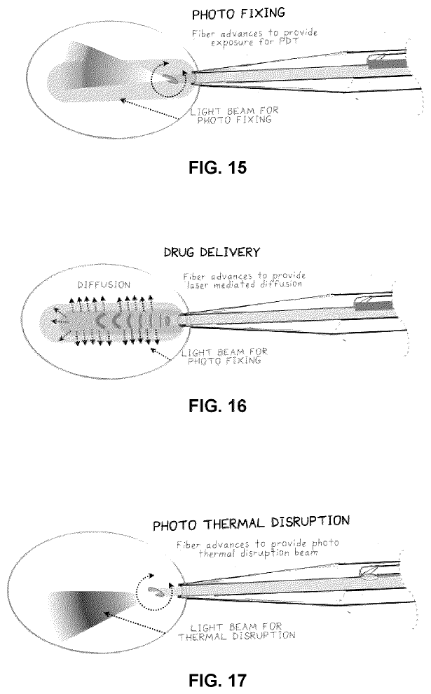

FIG. 15 illustrates an example embodiment in which the distal end of the optical fiber is angled or cone-shaped to allow exposure to a wider volume of tissue through translation and rotation of the fiber end either independently or simultaneously with additional PIRL irradiation. Such an implementation may facilitate the delivery of laser light of a suitable wavelengths, pulse durations, and time durations to modify the surroundings of the targeted site to prevent diffusion of the drug away from the target. This step may involve, for example, photorelease of removable group on a particle drug, such as photodynamic therapy (PDT) drugs but not limited to PDT drugs, to lead to chemical bonding in place at the target site, i.e., block and fix the drug to the target site. The action of the drug in the case of photodynamic therapy can be controlled by light exposure using the optical fiber that was employed for initial disruption of the target tissue, or, for example, a replacement optical fiber using the same path. Another means to exploit light for blocking drug diffusion involves coagulating the surrounding area or cauterizing by higher power irradiation to physically block diffusion to enable real time monitoring and changes in treatment to reduce cancer volumes or ensure completely eradication of the cancerous tissue.

In some example implementations, as illustrated in FIG. 16, after drug delivery, the PIRL radiation may be provided to contact the dispensed drug fluid and generate a photomechanical force that improves diffusion of the pharmaceutical agent by creating large pressure gradients within the inside of the tumor through photomechanical cavitation effects.

In another example embodiment, as illustrated in FIG. 17, an additional laser source is provided for photothermal treatment of the target tumor, as previously described.

In another example embodiment, the example embodiments described above may be configured to obtain a liquified tissue microbiopsy sample.

In other example embodiments, the optical fiber employed for the delivery of PIRL pulses may be couped to an external optical detection system that is configured to deliver, through the optical fiber, interrogating optical energy to the disrupted and liquified tissue, and to collect optical energy that is responsively emitted by the disrupted and liquified tissue. The disrupted and liquified tissue may reside beyond the distal end of the cannula. Alternatively, the disrupted and liquified tissue may reside within a distal portion of the cannula after retraction of the optical fiber to create a partial vacuum. Non-limiting example modalities for performing in-situ microbiopsy include spectroscopic methods such as Raman spectroscopy, fluorescence spectroscopy and frequency comb and laser induced breakdown spectroscopy. For example, Raman spectroscopy can be performed by employing the optical fiber used for PIRL disruption to also deliver excitation energy and collect backscattered Raman signals, which can be analyzed for biomarkers of disease or normal tissue. The very high excitation and thermal heating of tissue may also lead to light emission that provides a spectral signature or fingerprint of particular constituents of the tissue. Such an approach may be employed, for example, to determine whether or not the distal tip of the optical fiber resides within the tumor target (or another identifiable intracorporeal tissue region), and such analysis may be performed, for example, before, during or after the delivery of a given form of local therapy.

In other example embodiments, a fluidic channel employed for the delivery of a therapeutic agent, and/or an additional fluidic channel, can be interfaced with a pump (e.g. a mechanical pump or a syringe), and the pump can be controlled to aspirate one or more volumes of the disrupted and liquified tissue for biopsy analysis. This biopsy aspiration step may be performed before, during, or after the delivery of local therapy to the selected intracorporeal tissue region.

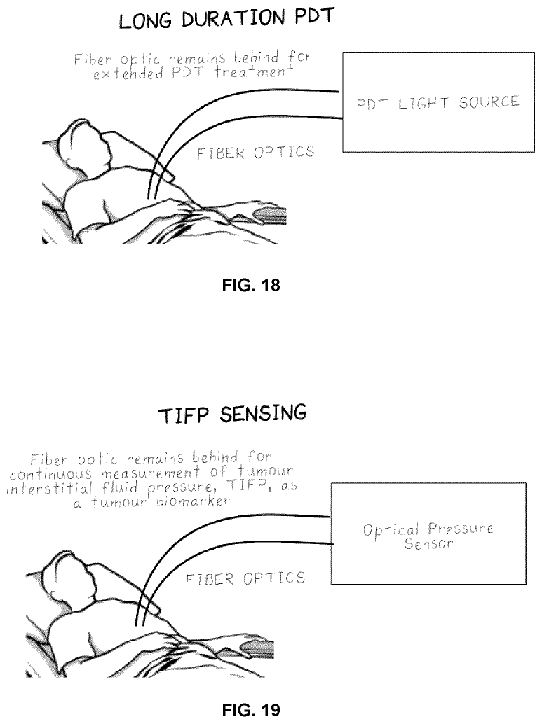

In another example embodiment, one or more optical fibers that were inserted into the subject may remain in place after the injection procedure, as illustrated in FIG. 18, for extended duration photo-dynamic therapy, PDT, via a PDT light source.

In another example embodiment, the optical fibers can include an optical pressure sensor for continuous measurement of IFP which can be a useful biomarker of the evolution of the tumor microenvironment and response to treatment, as illustrated in FIG. 19.

In another example embodiment, the system is configured so that the tumor tissue which is disrupted on contact with the laser energized fiber tip is purposely released into the tumor surroundings, by retraction of the needle or other means of flushing tissue from within the tumor or aspirating and reinjecting it outside the tumor, including into the vascular system so as to create an abscopal effect whereby the disrupted tissue contains tumor specific antigens that have not become damaged or denatured during disruption. This system can be deployed, for example, independently or in combination with drugs to activate the immune system, immune checkpoint inhibitors, T-cell, macrophage, dendritic cell, and innate immunity modulators.

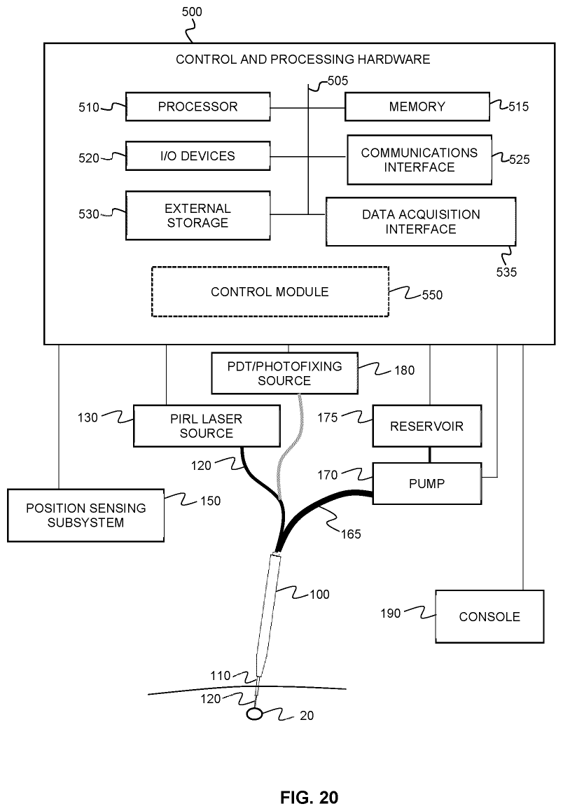

FIG. 20 illustrates an example system for performing PIRL-based local tissue disruption followed by direct local drug injection into the disrupted tissue. PIRL laser pulses are generated by a PIRL laser source 130, and the laser pulses are delivered through an optical fiber 120 that passes through a probe body 100 (handpiece) having a distal cannula region 110, with the optical fiber 120 being optionally extendable from the distal end of the cannula 110. The figure shows the insertable cannula 110 being inserted into a subject with the distal portion of the optical fiber 120 extended to irradiate and disrupt a tissue region 20.

The laser source 130 is operatively coupled or connectable to control and processing hardware 500 for control thereof. The example control and processing hardware 500 may include a processor 510, a memory 515, a system bus 505, one or more input/output devices 520, and a plurality of optional additional devices such as communications interface 525, external storage 530, and a data acquisition interface 535. In one example implementation, a display (not shown) may be employed to provide a user interface for facilitating input to control the operation of the system 500. The display may be directly integrated into a control and processing device (for example, as an embedded display), or may be provided as an external device (for example, an external monitor).

A reservoir 175 contains a fluid therapeutic agent (e.g., pharmaceutical compound, drug), and delivery of the drug to the tissue region, through the probe body 100, is achieved by action of a pump 170 that is controlled by control and processing system 500.

Position sensing and guidance of the cannula 110 and distal optical fiber 120 (or optical waveguide) is facilitated by position sensing subsystem 150, which is interfaced with the control and processing system 500.

The figure also shows the optional inclusion of an additional laser source that is also optically coupled (e.g., through a wavelength-multiplexing device or an optical coupler) to the optical fiber 120 or distal optical waveguide for the delivery of an additional form of optical radiation, such as a laser source suitable for photo fixing, photodynamic therapy, and/or photothermal disruption or thermally driven apoptosis.

The control and processing system 500 may include or be connectable to a console 190 that provides an interface for facilitating an operator to control the laser source 160. The console may include, for example, one or more input devices, such, but not limited to, a keypad, mouse, joystick, touchscreen, and may optionally include a display device.

The methods described herein, such as methods for controlling the sequence of operations of the local PIRL-based laser disruption and the subsequent fluid delivery, optionally controlling the extension and retraction of the optical fiber, and/or controlling one or more valves in fluid connection with the pump and/or reservoir, and other example methods described below, can be implemented via processor 510 and/or memory 515. As shown in FIG. 20, executable instructions represented as control module 550 are processed by control and processing hardware 500. Such executable instructions may be stored, for example, in the memory 515 and/or other internal storage.

The methods described herein can be partially implemented via hardware logic in processor 510 and partially using the instructions stored in memory 515. Some embodiments may be implemented using processor 510 without additional instructions stored in memory 515. Some embodiments are implemented using the instructions stored in memory 515 for execution by one or more microprocessors. Thus, the disclosure is not limited to a specific configuration of hardware and/or software.

It is to be understood that the example system shown in the figure is not intended to be limited to the components that may be employed in a given implementation. For example, the system may include one or more additional processors. Furthermore, one or more components of control and processing hardware 500 may be provided as an external component that is interfaced to a processing device. Furthermore, although the bus 505 is depicted as a single connection between all of the components, it will be appreciated that the bus 505 may represent one or more circuits, devices or communication channels which link two or more of the components. For example, the bus 505 may include a motherboard. The control and processing hardware 500 may include many more or less components than those shown.

Some aspects of the present disclosure can be embodied, at least in part, in software, which, when executed on a computing system, transforms an otherwise generic computing system into a specialty-purpose computing system that is capable of performing the methods disclosed herein, or variations thereof. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine-readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

The example embodiments described above can be employed for a wide variety of clinical applications. It will be understood that the aforementioned example therapeutic applications involving direct intratumoral injection and local chemotherapy are but one example implementation and are not intended to limit the scope of the present disclosure. It will be understood that the example embodiments may be employed for a wide variety of other applications, including, for example, the local delivery of fluids to tissues other than cancerous tissue, for example, for site selective drug treatments or microbiopsies for detection of cancer or other disease states.

In some example implementations, the example embodiments disclosed herein may be employed for intracranial applications including, but not limited to, local chemotherapy of brain metastases, neuromodulation and neurostimulation. For example, the advancement of a PIRL-based probe (e.g., an optical fiber or probe housing an optical fiber or other optical waveguide for delivery of PIRL pulses), via PIRL-based tissue disruption and liquification with minimal collateral damage along the entry path, may be employed to enter the brain with minimal collateral damage to surrounding tissue. Such a minimally invasive PIRL-based probe may be employed for the treatment of intracranial pathologies, e.g., via tissue disruption and liquification and/or thermal treatment. A probe configured to facilitate both PIRL tissue disruption and liquification and local fluid delivery, such as the example embodiments disclosed above or variations thereof, may be employed to facilitate atraumatic entry into the brain with the subsequent local delivery of therapeutic agents within the brain, optionally with PIRL-based tissue disruption and liquification and/or thermal treatment of internal tissues. Here the ability to enter the brain without tissue deformation, deflection from the optimal path to the region of interest, or collateral damage is a critical for the absolute minimally invasive procedure for brain tumors, lesions, biopsies and drug delivery.

While many of the example embodiments disclosed herein pertain to the local delivery of therapy to tumor tissue, it will be understood that the present example embodiments are not intended to be limited to the local treatment of tumor tissue and can alternatively be employed to delivery local therapy to any intracorporeal tissue region, or to provide internal access to any intracorporeal tissue for any minimally invasive treatment modality. For example, the present example methods may be employed to treat a wide range of pathologies associated with internal tissue, such as, but not limited to, localized disruption ablation of cardiac tissue to arrest fibrillation, and onset of heart attacks, removal of tissue around pinch nerves involved in chronic pain without the complication of scar tissue formation to reinjure nerves and reoccurrence of chronic pain, intravascular surgery, repair of vessels involved in internal bleeding such as strokes by thermal coagulation using the same form of beam delivery, removal of nasal and vocal cord polyps, creating openings for improved blood circulation, implants requiring the formation of cavity within tissue such as micro-cochlea implants, benign lesions, autoimmune diseases affecting specific, cardiovascular diseases such as atherosclerosis characterized by arterial fatty deposits, fibrotic diseases including pulmonary fibrosis and liver cirrhosis, and restenosis, where excessive tissue growth occurs after procedures like angioplasty, and nerve tissue disruption and orthopedic procedures.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Demonstration of Ultrasound Detection of Optical Fiber Tip Location via Detection of Photoacoustic Signals During Delivery of PIRL Pulses

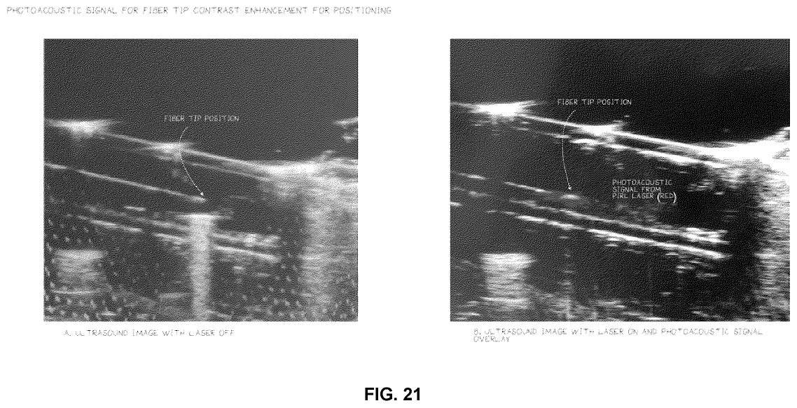

An optical fiber was imaged using a vivo 3100 high resolution ultrasound imaging system, as shown in FIG. 21. The left image shows the ultrasound image prior to the delivery of PIRL pulses by the optical fiber. The right image was obtained while the optical fiber was emitting PIRL pulses, and the image clearly shows the photoacoustic signal associated with PIRL-induced cavitation beyond the distal end of the optical fiber. The laser output was synchronized to the imaging system for timing gating of the imaging ultrasound signal and the photo acoustic signal generated by the laser firing in the liquid.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A system for performing local tissue disruption and liquification of an intracorporeal tissue region, said system comprising:
a pulsed infrared laser source configured to generate infrared laser pulses;
a laser pulse delivery assembly comprising:
an optical fiber optically coupled to said pulsed infrared laser source, such that the infrared laser pulses are delivered through said optical fiber to a distal tip of said optical fiber; and
a cannula configured to receive and mechanically support said optical fiber, such that said distal tip of said optical fiber is extendable to at least a distal end of said cannula for delivering the infrared laser pulses beyond said distal end of said cannula;
a navigation system configured to provide guidance during manipulation of said laser pulse delivery assembly to position said distal tip of said optical fiber proximal to the intracorporeal tissue region; and
control circuitry operatively coupled to said pulsed infrared laser source, said control circuitry being configured to perform operations comprising:
controlling said pulsed infrared laser source to emit the infrared laser pulses such that during penetration of the intracorporeal tissue region by said distal tip of said optical fiber, the infrared laser pulses have laser pulse properties including:
a wavelength selected such that absorption by a laser-irradiated tissue volume is predominantly due to excitation of vibrational modes of one or more constituents of the laser-irradiated tissue volume;
a pulse duration that is shorter than a first time duration required for thermal diffusion out of the laser-irradiated tissue volume and shorter than a second time duration required for a thermally driven expansion of the laser-irradiated tissue volume;
a pulse fluence and the pulse duration resulting in a peak pulse intensity below a threshold for ionization-driven tissue disruption and liquification to occur within the laser-irradiated tissue volume; and
wherein the pulse fluence is sufficiently high to cause local tissue disruption and liquification of the laser-irradiated tissue volume;
said pulsed infrared laser source thereby being controlled to facilitate local tissue disruption and liquification during penetration of said distal tip of said optical fiber into the intracorporeal tissue region, avoiding substantial deformation of the intracorporeal tissue region and facilitating positioning of said distal tip within the intracorporeal tissue region.

2. The system according to claim 1 wherein said control circuitry is configured to control said pulsed infrared laser source to deliver the infrared laser pulses with the laser pulse properties during manipulation of said laser pulse delivery assembly to position said distal tip of said optical fiber proximal to the intracorporeal tissue region, thereby locally disrupting tissue residing adjacent to said distal tip of said optical fiber while said distal tip of said optical fiber is moved through tissue toward the intracorporeal tissue region, avoiding substantial tissue deformation and facilitating positioning of said distal tip proximal to the intracorporeal tissue region.

3. The system according to claim 1 wherein said distal tip of said optical fiber is extendable beyond said distal end of said cannula to facilitate penetration of the intracorporeal tissue region by said distal tip of said optical fiber.

4. The system according to claim 1 wherein said control circuitry is further configured, after extension of said distal tip of said optical fiber into the intracorporeal tissue region, to control said pulsed infrared laser source to emit the infrared laser pulses with a reduced pulse fluence below a threshold for local tissue disruption and liquification, the reduced pulse fluence being sufficiently high to deliver thermal therapy within the intracorporeal tissue region for inducing apoptosis.

5. The system according to claim 1 further comprising an additional laser source optically coupled to said optical fiber, said additional laser source being configured to generate laser energy suitable for providing thermal therapy to the intracorporeal tissue region, wherein said control circuitry is further configured, after extension of said distal tip of said optical fiber into the intracorporeal tissue region, to control said additional laser source to emit the laser energy for inducing apoptosis within the intracorporeal tissue region.

6. The system according to claim 1 further comprising an optical detection system optically coupled to said optical fiber, said optical detection system being configured to deliver interrogating optical energy to tissue disrupted and liquified by the infrared laser pulses, and to detect optical energy responsively emitted by the disrupted and liquified tissue.

7. The system according to claim 1 wherein said laser pulse delivery assembly further comprises a liquid delivery conduit in flow communication with said distal end of said cannula;
said system further comprising a liquid delivery pump configured to deliver a liquid therapeutic agent to said liquid delivery conduit; and
wherein said control circuitry is operatively coupled to said liquid delivery pump, and wherein said control circuitry is further configured, after extension of said distal end of said cannula into the intracorporeal tissue region, to perform operations comprising:
controlling said liquid delivery pump to dispense the liquid therapeutic agent within the intracorporeal tissue region.

8. The system according to claim 7 wherein said cannula comprises a primary lumen through which said optical fiber is extendable, and wherein said liquid delivery conduit is provided as a side lumen of said cannula, said side lumen intersecting said primary lumen at an internal port residing within a distal region of said cannula, such that the liquid therapeutic agent residing in said liquid delivery conduit is brought into flow communication with said primary lumen, for dispensing the liquid therapeutic agent beyond said distal end of said cannula, after retraction of said distal tip of said optical fiber to a location that is proximal relative to said internal port.

9. The system according to claim 7 wherein said control circuitry is configured to control said liquid delivery pump to deliver the liquid therapeutic agent within the intracorporeal tissue region after having previously delivered thermal therapy to the intracorporeal tissue region.

10. The system according to claim 7 wherein the liquid therapeutic agent comprises a photodynamic therapy agent, said system further comprising a photodynamic excitation laser source optically coupled to said optical fiber, said photodynamic excitation laser source being configured to generate photodynamic laser energy suitable for causing photodynamic activation of the photodynamic therapy agent, wherein said control circuitry is further configured, after dispensing of the liquid therapeutic agent into the intracorporeal tissue region, to control said photodynamic excitation laser source to emit the photodynamic laser energy for activating the photodynamic therapy agent.

11. The system according to claim 1 wherein said laser pulse delivery assembly further comprises a microbiopsy aspiration conduit in flow communication with a lumen of said cannula;
said system further comprising a microbiopsy aspiration pump configured to cause a reduction in pressure within said microbiopsy aspiration conduit; and
wherein said control circuitry is operatively coupled to said microbiopsy aspiration pump, and wherein said control circuitry is further configured, after extension of said distal end of said cannula into the intracorporeal tissue region and local disruption and liquification of tissue within the intracorporeal tissue region, to perform operations comprising:
controlling said microbiopsy aspiration pump to aspirate a liquified tissue sample within said lumen of said cannula.

12. The system according to claim 1 wherein said laser pulse delivery assembly further comprises an aspiration conduit in flow communication with a distal region of said cannula;
said system further comprising an aspiration pump configured to cause a reduction in pressure within said aspiration conduit; and
wherein said control circuitry is operatively coupled to said aspiration pump, and wherein said control circuitry is further configured, during local disruption and liquification of tissue, to perform operations comprising:
controlling said aspiration pump to aspirate liquified tissue within said aspiration conduit.

13. The system according to claim 1 wherein said navigation system comprises an ultrasound imaging system, and wherein said ultrasound imaging system is configured to display, on a user interface, a location of said distal tip of said optical fiber, the location being determined based on detection of photoacoustic signals generated at said distal tip during delivery of the infrared laser pulses with the laser pulse properties.

14. The system according to claim 1 wherein a distal region of said cannula is tapered, such that an outer diameter of said cannula reduces in a distal direction toward said distal end of said cannula.

15. The system according to claim 1 wherein a diameter of said cannula, at said distal end of said cannula, exceeds a diameter of said optical fiber by less than 10% of the diameter of said optical fiber.

16. The system according to claim 1 wherein said distal end of said cannula is beveled.

17. The system according to claim 1 wherein said distal tip of said optical fiber is beveled, such that the infrared laser pulses are emitted at an oblique angle relative to a longitudinal axis of said optical fiber.

18. The system according to claim 17 wherein a bevel angle of said optical fiber lies within 10% of a bevel angle of said distal end of said cannula.

19. The system according to claim 17 wherein said optical fiber is rotatable relative to the intracorporeal tissue region, and wherein said control circuitry is further configured, after extension of said distal tip of said optical fiber into the intracorporeal tissue region, to control said pulsed infrared laser source to emit the infrared laser pulses with the laser pulse properties during rotation of said optical fiber to facilitate local disruption and liquification over an expanded volume within the intracorporeal tissue region.

20. The system according to claim 1 further comprising a steering means for steering one or both of said cannula and said optical fiber.

21. The system according to claim 1 wherein said laser pulse delivery assembly comprises one or more additional optical fibers, such that said optical fiber and said one or more additional optical fibers form an optical fiber bundle, and wherein said optical fiber bundle is optically coupled to said pulsed infrared laser source, such that the infrared laser pulses are delivered through said optical fiber bundle to a distal end of said optical fiber bundle.

22. The system according to claim 21 wherein at least two optical fibers of said optical fiber bundle have beveled distal tips configured to direct the infrared laser pulses in different directions.

23. A method of performing local tissue disruption and liquification of an intracorporeal tissue region, the method comprising:
while extending an optical fiber within a body, such that a distal tip of the optical fiber penetrates an intracorporeal tissue region, delivering, through the optical fiber, infrared laser pulses having laser pulse properties including:
a wavelength selected such that absorption by a laser-irradiated tissue volume is predominantly due to excitation of vibrational modes of one or more constituents of the laser-irradiated tissue volume;
a pulse duration that is shorter than a first time duration required for thermal diffusion out of the laser-irradiated tissue volume and shorter than a second time duration required for a thermally driven expansion of the laser-irradiated tissue volume;
a pulse fluence and the pulse duration resulting in a peak pulse intensity below a threshold for ionization-driven tissue disruption and liquification to occur within the laser-irradiated tissue volume; and
wherein the pulse fluence is sufficiently high to cause local tissue disruption and liquification of the laser-irradiated tissue volume;
the infrared laser pulses thereby causing local tissue disruption and liquification during penetration of the distal tip of the optical fiber into the intracorporeal tissue region, avoiding substantial deformation of the intracorporeal tissue region and facilitating positioning of said distal tip within the intracorporeal tissue region.

24. The method according to claim 23 wherein, prior to penetrating the intracorporeal tissue region, the infrared laser pulses are delivered during manipulation of the optical fiber to position the distal tip of the optical fiber proximal to the intracorporeal tissue region, thereby locally disrupting tissue residing adjacent to the distal tip of the optical fiber while the distal tip of said optical fiber is moved through tissue toward the intracorporeal tissue region, avoiding substantial tissue deformation and facilitating positioning of the distal tip proximal to the intracorporeal tissue region.

25. The method according to claim 23 wherein, after extension of the distal tip of the optical fiber into the intracorporeal tissue region, additional infrared laser pulses with a reduced pulse fluence below a threshold for local tissue disruption and liquification are delivered by the optical fiber, the reduced pulse fluence being sufficiently high to deliver thermal therapy within the intracorporeal tissue region for inducing apoptosis.

26. The method according to claim 23 further comprising employing an additional laser source optically coupled to the optical fiber, to deliver laser energy suitable for providing thermal therapy to the intracorporeal tissue region to induce apoptosis within the intracorporeal tissue region.

27. The method according to claim 23 further comprising employing an optical detection system optically coupled to the optical fiber to deliver interrogating optical energy to tissue disrupted and liquified by the infrared laser pulses, and to detect optical energy responsively emitted by the disrupted and liquified tissue.

28. The method according to claim 23 wherein the distal tip of the optical fiber is extended beyond a distal end of a cannula to facilitate penetration of the intracorporeal tissue region.

29. The method according to claim 28 wherein the cannula further comprises a liquid delivery conduit in flow communication a distal end of the cannula, the liquid delivery conduit being primed with a liquid therapeutic agent, the method further comprising, after having penetrated the intracorporeal tissue region with the distal tip of the optical fiber:
extending the distal end of the cannula into the intracorporeal tissue region;
retracting the optical fiber into the cannula; and
dispensing the liquid therapeutic agent within the intracorporeal tissue region.

30. The method according to claim 29 wherein the cannula comprises a primary lumen through which the optical fiber is extendable, and wherein the liquid delivery conduit is provided as a side lumen of the cannula, the side lumen intersecting the primary lumen at an internal port residing within a distal region of the cannula, such that the liquid therapeutic agent residing in the liquid delivery conduit is brought into flow communication with the primary lumen, for dispensing the liquid therapeutic agent beyond the distal end of the cannula, after retraction of the distal tip of the optical fiber to a location that is proximal relative to the internal port.

31. The method according to claim 29 further comprising delivering the liquid therapeutic agent within the intracorporeal tissue region after having previously delivered thermal therapy to the intracorporeal tissue region.

32. The method according to claim 29 wherein the liquid therapeutic agent comprises a photodynamic therapy agent, the method further comprising employing a photodynamic excitation laser source optically coupled to the optical fiber to deliver photodynamic laser energy suitable for causing photodynamic activation of the photodynamic therapy agent.

33. The method according to claim 28 further comprising, after having penetrated the intracorporeal tissue region with the distal tip of the optical fiber:
extending the distal end of the cannula into the intracorporeal tissue region;
employing a pump to lower a pressure in a lumen of the cannula to aspirate a liquified tissue sample.

34. The method according to claim 23 wherein penetration of the intracorporeal tissue region by the optical fiber is guided by an ultrasound imaging system, and wherein the ultrasound imaging system is configured to display, on a user interface, a location of the distal tip of the optical fiber, the location being determined based on detection of photoacoustic signals generated at the distal tip during delivery of the infrared laser pulses with the laser pulse properties.

35. The method according to claim 23 wherein said distal tip of said optical fiber is beveled, such that the infrared laser pulses are emitted at an oblique angle relative to a longitudinal axis of the optical fiber.

36. The method according to claim 35 further comprising, after extension of the distal tip of the optical fiber into the intracorporeal tissue region, emitting additional infrared laser pulses with the laser pulse properties during rotation of the optical fiber to facilitate local disruption and liquification over an expanded volume within the intracorporeal tissue region.

37. The method according to claim 23 wherein the intracorporeal tissue region is a tumor.
